(19) United States
(12) Patent Application Publication
(10) Pub. No.: US 2025/0388573 A1
(43) Pub. Date: Dec. 25, 2025

(54) FUSED BICYCLIC EGFR INHIBITORS AND METHODS OF USE THEREOF

(71) Applicant: Dana-Farber Cancer Institute, Inc., Boston, MA (US)

(72) Inventors: David A. Scott, Boston, MA (US); Thomas Gero, Boston, MA (US)

(21) Appl. No.: 18/877,585

(22) PCT Filed: Jun. 20, 2023
(86) PCT No.: PCT/US2023/068726

§ 371 Date: Dec. 20, 2024

Related U.S. Application Data
(60) Provisional application No. 63/366,732, filed on Jun. 21, 2022.

(51) Int. Cl. C07D 417/14; A61K 31/506; A61K 31/517; A61K 31/536; A61K 31/5365; A61K 31/5377; C07D 413/14; C07D 417/12; C07D 498/04

(52) U.S. Cl. CPC C07D 417/14; A61K 31/506; A61K 31/517; A61K 31/536; A61K 31/5365; A61K 31/5377; C07D 413/14; C07D 417/12; C07D 498/04

(57) ABSTRACT

The disclosure relates to fused bicyclic compounds comprising ring cores such as 2H-benzo[e][1,3]oxazin-4(3H)-one, 2,3-dihydrothieno[2,3-e][1,3]oxazin-4-one and 2,3-dihydrothieno[3,2-e][1,3]oxazin-4-one, that act as allosteric inhibitors of epidermal growth factor receptor (EGFR); pharmaceutical compositions comprising the compounds, optionally in combination with a second active agent; and methods of treating or preventing kinase-mediated disorders, including cancer and other proliferation diseases.

### RELATED APPLICATIONS

This application is related to U.S. Provisional Application No. 63/366,732, filed on Jun. 21, 2022, the content of which is incorporated by reference in its entirety.

### STATEMENT REGARDING FEDERALLY SPONSORED RESEARCH OR DEVELOPMENT

This invention was made with government support under Grant No. 5R01CA201049 awarded by the National Institutes of Health (NIH). The government has certain rights in the invention.

### BACKGROUND

The epidermal growth factor receptor (EGFR, Erb-B1) belongs to a family of receptor tyrosine kinases that mediate the proliferation, differentiation, and survival of normal and malignant cells (Arteaga, C. L., J. Clin. Oncol. 19, 2001, 32-40). Deregulation of EGFR has been implicated in many types of human cancer, with overexpression of the receptor present in at least 70% of human cancers (Seymour, L. K., Curr. Drug Targets 2, 2001, 117-133), including non-small lung cell carcinomas, breast cancers, gliomas, squamous cell carcinomas of the head and neck, and prostate cancer (Raymond, E., et al., Drugs 60 (Suppl. 1), 2000, 15-23, discussion 41-2; Salomon, D. S., et al., Crit. Rev. Oncol. Hematol. 19, 1995, 183-232; Voldborg B. R., et al., Ann. Oncol. 8, 1997, 1197-1206). EGFR has therefore emerged as an attractive target for the design and development of diagnostic and therapeutic agents that can specifically bind and inhibit the receptor's tyrosine kinase activity and signal transduction pathway in cancer cells. The two most common EGFR activating mutations in non-small cell lung cancer (NSCLC) patients are deletions in exon 19 (del19) and the L858R point mutation.

The reversible EGFR inhibitors gefitinib and erlotinib are effective clinical therapies for EGFR mutant advanced non-small cell lung cancer (NSCLC) patients (Mok, T. S., et al., N. Engl. J. Med. 361, 2009, 947-57; Paez, J. G., et al., Science 304, 2004, 1497-500; Lynch, T. J., et al., N. Engl. J. Med. 350, 2004, 2129-39; Rosell, R., et al., Lancet Oncol. 13, 2012, 239-46). However, most patients will develop disease progression following treatment with these drugs. The most common mechanism of acquired resistance, in 60% of patients, is a secondary mutation in EGFR at position T790 (T790M) (Yu, H. A., et al., Clin. Cancer Res. 19, 2013, 2240-7). This mutation leads to an increase in ATP affinity, thus making it more difficult for reversible EGFR tyrosine kinase inhibitors (TKIs) gefitinib and erlotinib to bind the EGFR TKI domain (Yun C. H., et al., Proc. Natl. Acad. Sci. USA 105, 2008, 2070-5).

Covalent EGFR inhibitors were developed to address resistance conferred by the EGFR T790M mutation. One such covalent EGFR inhibitor is afatinib, which is potent against both mutant and wild type (WT) EGFR (Li, D.; et al., Oncogene 27, 2008 (4702-2711). Inhibition of WT EGFR, however, leads to toxicities including skin rash and diarrhea, which can be dose-limiting in the clinic. (Yap, T. A.; et al., J Clin Onc, 28, 2010 (3965-3972).

Third generation covalent EGFR inhibitors with selectivity for mutant EGFR over WT EGFR overcome the limitations of afatinib and demonstrate improved clinical efficacy and tolerability (Cross, D. E. A.; et al., Cancer Discov. 4, 2014 (1046-1061). Osimertinib was first approved for patients who had developed resistance to reversible inhibitors through the T790M mutation and has now also been approved for first line therapy. Despite addressing the major resistance mechanisms of the first-generation inhibitors, resistance to osimertinib also emerges (Schmid, S., et al., Lung Cancer 147, 2020, 123-129). Mechanisms of resistance include the C797S mutation, which prevents osimertinib forming a covalent bond to the cysteine residue (Thress, K. T., et al., Nature Med. 21, 2015, 560-562). This resistance results in either a triple mutant variant (del19/T790M/C797S or L858R/T790M/C797S) or a double mutant in the case of first line therapy (e.g., L858R/C797S), which cannot be successfully treated with the current set of approved EGFR inhibitors.

Therefore, there remains a need for new targeted therapies for NSCLC patients who have developed resistance to existing inhibitors.

### SUMMARY

In an aspect, provided herein is a compound of Formula I:

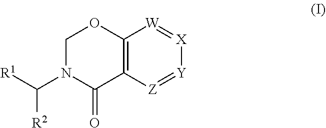

or a pharmaceutically acceptable salt thereof.

In another aspect, provided herein is a compound of Formula III:

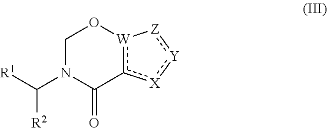

or a pharmaceutically acceptable salt thereof.

In another aspect, provided herein is a pharmaceutical composition comprising a compound disclosed herein, or a pharmaceutically acceptable salt thereof, and at least one pharmaceutically acceptable carrier.

In yet another aspect, provided herein is a method of treating cancer in a subject in need thereof, comprising administering to the subject a therapeutically effective amount of a compound disclosed herein.

In still another aspect, provided herein is a method of inhibiting a kinase in a subject in need thereof, comprising administering to the subject a therapeutically effective amount of a compound disclosed herein.

In an aspect, provided herein is a method of treating or preventing a kinase-mediated disorder in a subject in need thereof, comprising administering to the subject a therapeutically effective amount of a compound disclosed herein.

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with wild-type or mutant EGFR an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “prodrug” refers to a precursor compound that will undergo metabolic activation in vivo to produce an active drug. Thus, for example, a prodrug of a compound provided herein will, when administered to a subject, undergo metabolic activation to generate the compound.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the disclosure and a co-agent, are both administered to a patient simultaneously in the form of a single entity or dosage. The term “non-fixed combination” means that the active ingredients, e.g. a compound of the disclosure and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “EGFR” refers to epidermal growth factor receptor (alternately referred to as ErbB-1 or HER1) and may refer to the wild-type receptor or to a receptor containing one or more mutations.

As used herein, the term “HER” or “Her” refers to members of the ErbB receptor tyrosine kinase family, including EGFR, ERBB2, HER3, and HER4.

As used herein, the term “allosteric site” refers to a site on EGFR other than the ATP binding site, such as that characterized in a crystal structure of EGFR. An “allosteric site” can be a site that is close to the ATP binding site, such as that characterized in a crystal structure of EGFR. For example, one allosteric site includes one or more of the following amino acid residues of epidermal growth factor receptor (EGFR): Lys745, Leu788, Ala743, Cys755, Leu777, Phe856, Asp855, Met766, Ile759, Glu762, and/or Ala763.

As used herein, the term “agent that prevents EGFR dimer formation,” or iterations thereof, refers to an agent that prevents dimer formation in which the C-lobe of the “activator” subunit impinges on the N-lobe of the “receiver” subunit. Examples of agents that prevent EGFR dimer formation include, but are not limited to, cetuximab, trastuzumab, panitumumab, and Mig6.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert butyl, pentyl, neopentyl, and hexyl. Other examples of C₁-C₆ alkyl include ethyl, methyl, isopropyl, isobutyl, n-pentyl, and n-hexyl.

As used herein, the term “haloalkyl” refers to an alkyl group, as defined above, substituted with one or more halo substituents, wherein alkyl and halo are as defined herein. Haloalkyl includes, by way of example, chloromethyl, trifluoromethyl, bromoethyl, chlorofluoroethyl, and the like.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkylamine” refers to the group —NH-alkyl, wherein alkyl is as defined herein. Alkylamine includes, by way of example, methylamine, ethylamine, isopropylamine, n-propylamine, n-butylamine, sec-butylamine, t-butylamine and the like. As used herein, the term “haloalkoxy” refers to the group —O-haloalkyl, wherein haloalkyl is as defined herein. Haloalkoxy includes, by way of example, chloromethoxy, trifluoromethoxy, bromoethoxy, chlorofluoroethoxy, and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “alkynyl” refers to a monovalent group derived from a hydrocarbon moiety containing, in certain embodiments, from two to six, or two to eight carbon atoms having at least one carbon-carbon triple bond. The alkynyl group may or may not be the point of attachment to another group. The term “alkynyl” includes, but is not limited to, ethynyl, 1-propynyl, 1-butynyl, heptynyl, octynyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, cycloalkyl is 3-10 membered cycloalkyl. In another embodiment, cycloalkyl is 3-6 membered cycloalkyl.

As used herein, the term “cycloalkenyl” means a non-aromatic carbocyclic system that is partially saturated having 1, 2 or 3 rings wherein such rings may be fused, and wherein at least one ring contains an sp² carbon-carbon bond. The term “cycloalkenyl” includes, but is not limited to, cyclopropenyl, cyclobutenyl, cyclopentenyl, cyclohexenyl, bicyclo[3.1.0]hexenyl, spiro[3.3]-heptanenyl, and bicyclo[1.1.1]pentenyl. In an embodiment, cycloalkenyl is 3-10 membered cycloalkyl. In another embodiment, cycloalkenyl is 4-7 membered cycloalkyl.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]hexanyl, 6-azabicyclo[3.1.1]heptanyl, 2-azabicyclo[2.2.1]heptanyl, 3-aza-bicyclo[3.1.1]heptanyl, 2-azabicyclo[3.1.1]heptanyl, 3-azabicyclo[3.1.0]hexanyl, 2-azabicyclo-[3.1.0]hexanyl, 3-azabicyclo[3.2.1]octanyl, 8-azabicyclo[3.2.1]octanyl, 3-oxa-7-azabicyclo[3.3.1]-nonanyl, 3-oxa-9-azabicyclo[3.3.1]nonanyl, 2-oxa-5-azabicyclo[2.2.1]heptanyl, 6-oxa-3-aza-bicyclo[3.1.1]heptanyl, 2-azaspiro[3.3]heptanyl, 2-oxa-6-azaspiro[3.3]heptanyl, 2-oxaspiro-[3.3]heptanyl, 2-oxaspiro[3.5]nonanyl, 3-oxaspiro[5.3]nonanyl, and 8-oxabicyclo[3.2.1]octanyl. In an embodiment, heterocycloalkyl is 3-10 membered heterocycloalkyl. In another embodiment, heterocycloalkyl is 5-7 membered heterocyclyl.

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]-triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, heteroaryl is 5-10 membered heteroaryl. In another embodiment, heteroaryl is 5-6 membered heteroaryl.

It is to be understood that if an aryl, heteroaryl, cycloalkyl, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

Provided herein are compounds that are allosteric inhibitors of epidermal growth factor receptor (EGFR) useful in the treatment of kinase-mediated disorders, including cancer and other proliferation diseases.

In an aspect, provided herein is a compound of Formula I:

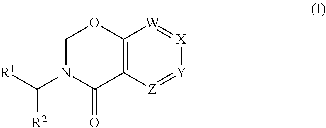

or a pharmaceutically acceptable salt thereof;
wherein:
W and Z are each, independently, N, CH, C-halo, C—(C₁-C₃ alkyl), or C—(C₁-C₃ alkoxy);
X and Y are each, independently, N, CH, or CR³;
provided that at least one of W, X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl, all of which are optionally substituted with one, two, or three R⁸;
R² is selected from the group consisting of 6-10 membered aryl, 5-10 membered heteroaryl, 3-10 membered heterocycloalkyl, and 3-10 membered cycloalkyl, all of which are optionally substituted with one, two, or three R⁶;
R³ is independently, at each occurrence, selected from the group consisting of halogen, OR⁴, NR⁴R⁴, SO₂R⁴, SO₂NHR⁴, NHSO₂R⁴, C(O)OR⁴, C(O)NHR⁴, C(O)R⁴, C₁-C₆ alkyl, C2-C₆ alkenyl, C2-C₆ alkynyl, 3-7 membered cycloalkyl, C4-C7 cycloalkenyl, C6-C10 aryl, 5-6 membered heteroaryl, and 5-7 membered heterocyclyl, wherein alkyl, alkenyl, or alkynyl are each optionally substituted one, two, or three times with R⁴, and wherein aryl, heteroaryl, or heterocyclyl are each optionally substituted one, two, or three times with R⁵;
R⁴ is independently, at each occurrence, selected from the group consisting of H, (CH₂)₀₋₃—(C₃-C₇ cycloalkyl), (CH₂)₀₋₃—(C₄-C₇ cycloalkenyl), (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁵;
R⁵ is independently, at each occurrence, selected from the group consisting of C₁-C₆ alkyl, C₁-C₆ haloalkyl, C₁-C₆ alkoxy, C₁-C₆ haloalkoxy, C₁-C₃ alkylamine, 3-10 membered cycloalkyl, halogen, COOH, C(O)O(C₁-C₆ alkyl), O(CH₂)₁₋₃—OH, NH₂, NH(C₁-C₆ alkyl), N(C₁-C₆ alkyl)₂, OH, CN, (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN;
alternatively, two R⁶, together with the atoms to which they are attached, can form 5-10 membered heteroaryl, 6-10 membered aryl, 3-10 membered heterocycloalkyl, or 3-10 membered cycloalkyl;
R⁷ is independently, at each occurrence, selected from the group consisting of C₁-C₆ alkyl, C₁-C₆ haloalkyl, C₁-C₆ alkoxy, C₁-C₆ haloalkoxy, halogen, NH₂, NH(C₁-C₆ alkyl), N(C₁-C₆ alkyl)₂, SO₂NH₂, SO₂NH(C₁-C₆ alkyl), SO₂N(C₁-C₆ alkyl)₂, (CH₂)₁₋₂—OH, C(O)(CH₂)₁₋₂—OH, C(O)(C₁-C₆ alkyl), and C(O)O(C₁-C₆ alkyl);
alternatively, two R⁷, together with the atoms to which they are attached, can form 5-membered heteroaryl, 6-10 membered aryl, 3-10 membered heterocycloalkyl, or 3-10 membered cycloalkyl;
R⁸ is independently, at each occurrence, selected from the group consisting of hydrogen, C₁-C₃ alkyl, C₁-C₃ haloalkyl, C₁-C₃ alkoxy, C₁-C₃ haloalkoxy, C₁-C₃ alkylamine, 3-6 membered cycloalkyl, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN; and
R⁹ is selected from the group consisting of 6-10 membered aryl, 5-10 membered heteroaryl, 3-10 membered heterocycloalkyl, and 3-10 membered cycloalkyl.

In an embodiment of Formula I, or a pharmaceutically acceptable salt thereof,
W and Z are each, independently, N, C-halo, or CH;
X and Y are each, independently, CH or CR³;
provided that at least one of W, X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl;
R² is selected from the group consisting of 6-10 membered aryl and 5-10 membered heteroaryl, both of which are optionally substituted with one, two, or three R⁶;
R³ is independently, at each occurrence, selected from the group consisting of C₆-C₁₀ aryl, 5-6 membered heteroaryl, and 5-7 membered heterocyclyl, wherein aryl, heteroaryl, or heterocyclyl are each optionally substituted one, two, or three times with R⁵;
R⁵ is independently, at each occurrence, selected from the group consisting of 3-10 membered cycloalkyl, (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, and OH;
R⁷ is independently, at each occurrence, selected from the group consisting of C₁-C₆ alkyl, C₁-C₆ haloalkyl, C₁-C₆ alkoxy, and halogen;
R⁸ is selected from the group consisting of hydrogen, C₁-C₃ alkyl, C₁-C₃ haloalkyl, and C₁-C₃ alkoxy; and
R⁹ is 5-10 membered heteroaryl.

In an embodiment, X is CH or CR³. In another embodiment, X is CH. In yet another embodiment, X is CR³.

In still another embodiment, Y is CH or CR³. In an embodiment, Y is CH. In another embodiment, Y is CR³.

In an embodiment, no more than two of W, X, Y, or Z is N. In another embodiment, no more than one of W, X, Y, or Z is N. In yet another embodiment, only one of X or Y is CR³. In still another embodiment, X is CR³. In another embodiment, Y is CR³.

In another embodiment, the compound of Formula I is a compound of Formula IIa or IIb:

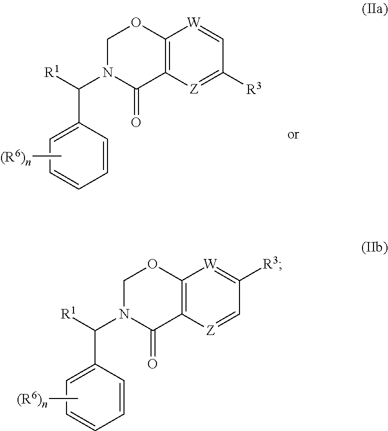

or a pharmaceutically acceptable salt thereof;
wherein n is 0, 1, or 2.

In yet another embodiment, the compound of Formula I is a compound of Formula IIa. In still another embodiment, the compound of Formula I is a compound of Formula IIb. In an embodiment, n is 1. In another embodiment, n is 1. In yet another embodiment, n is 2.

In an embodiment, W is N or CH. In another embodiment, W is N. In yet another embodiment, W is CH.

In still another embodiment, Z is CH or C-halo. In an embodiment, Z is CH. In another embodiment, Z is C-halo.

In yet another embodiment, R¹ is C(O)NHR⁹ or 5-10 membered heteroaryl.

In still another embodiment, R¹ is selected from the group consisting of:

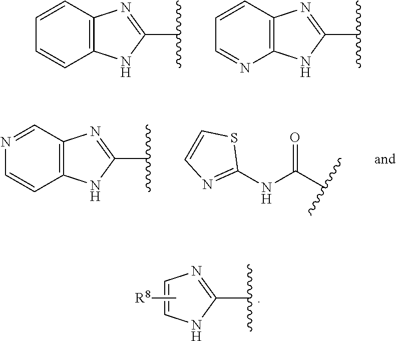

In an embodiment, R⁸ is C₁-C₃ alkyl.

In another embodiment, R¹ is

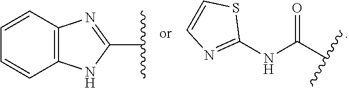

In yet another embodiment,
R³ is independently, at each occurrence, C₆-C₁₀ aryl optionally substituted one, two, or three times with R⁵;
R⁵ is independently, at each occurrence, selected from the group consisting of (CH₂)₀₋₃-(5-7 membered heterocyclyl) optionally substituted one, two, or three times with R⁷; and
R⁷ is independently, at each occurrence, selected from the group consisting of C₁-C₆ alkyl.

In still another embodiment, R³ is independently, at each occurrence, selected from the group consisting of

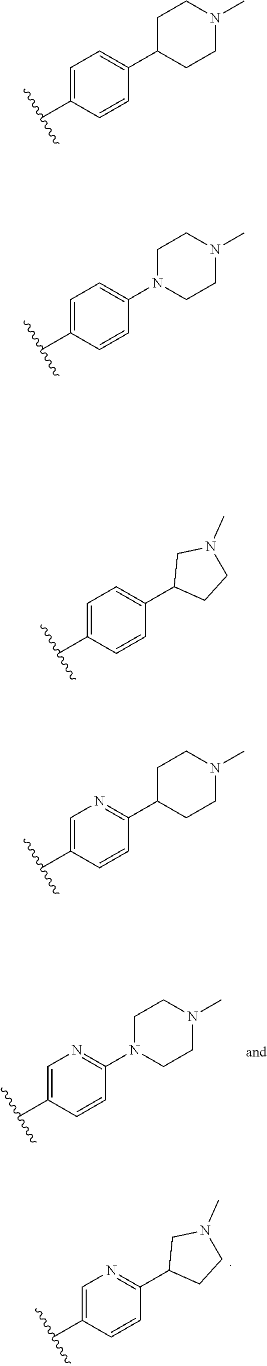 [SMILES: *c1ccc(C2CCN(C)CC2)cc1 ; *c1ccc(N2CCN(C)CC2)cc1 ; *c1ccc(C2CCN(C)C2)cc1 ; *c1ccc(C2CCN(C)CC2)nc1 ; *c1ccc(N2CCN(C)CC2)nc1 ; *c1ccc(C2CCN(C)C2)nc1]

In an embodiment, R³ is independently, at each occurrence, selected from the group consisting of

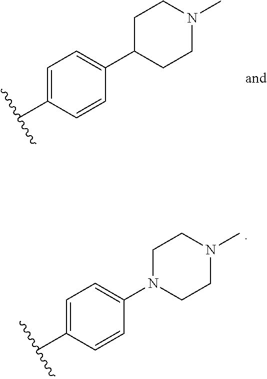 [SMILES: *c1ccc(C2CCN(C)CC2)cc1 ; *c1ccc(N2CCN(C)CC2)cc1]

In another embodiment, the compound of Formula I is a compound of Formula IIa or IIb, or a pharmaceutically acceptable salt thereof,
wherein
W is N or CH;
Z is CH or C-halo;
R¹ is selected from the group consisting of C(O)NHR⁹,

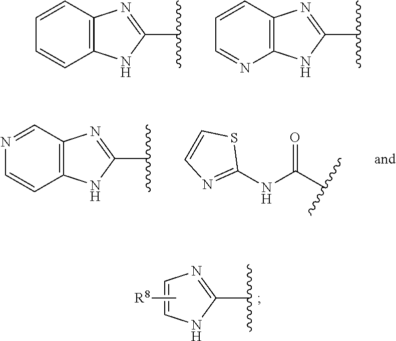

R⁸ is C₁-C₃ alkyl; and
R⁹ is 5-membered heteroaryl.

In another embodiment, the compound of Formula I is selected from the group consisting of a compound in Table 1.

TABLE 1

| Compound | |
|---|---|
| No. | Structure |
| 001 | 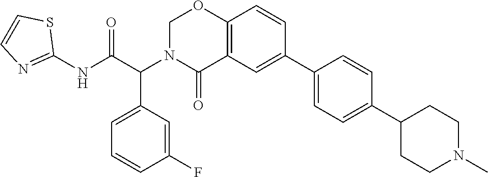 [SMILES: CN1CCC(c2ccc(-c3ccc4c(c3)C(=O)N(C(C(=O)Nc3nccs3)c3cccc(F)c3)CO4)cc2)CC1] |
| 002 | 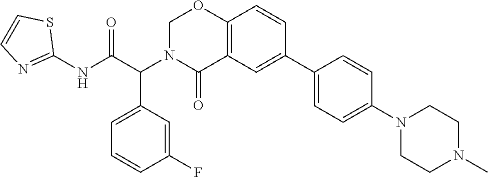 [SMILES: CN1CCN(c2ccc(-c3ccc4c(c3)C(=O)N(C(C(=O)Nc3nccs3)c3cccc(F)c3)CO4)cc2)CC1] |
| 003 | 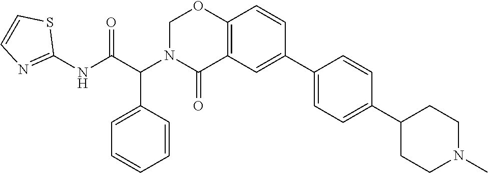 [SMILES: CN1CCC(c2ccc(-c3ccc4c(c3)C(=O)N(C(C(=O)Nc3nccs3)c3ccccc3)CO4)cc2)CC1] |
| 004 | 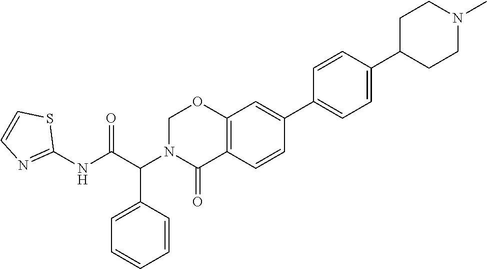 [SMILES: CN1CCC(c2ccc(-c3ccc4c(c3)OCN(C(C(=O)Nc3nccs3)c3ccccc3)C4=O)cc2)CC1] |
| 005 | 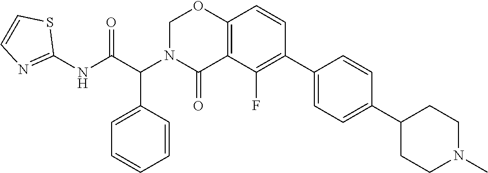 [SMILES: CN1CCC(c2ccc(-c3ccc4c(c3F)C(=O)N(C(C(=O)Nc3nccs3)c3ccccc3)CO4)cc2)CC1] |
| 006 | 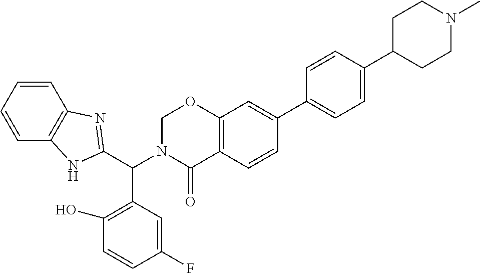 [SMILES: CN1CCC(c2ccc(-c3ccc4c(c3)OCN(C(c3nc5ccccc5[nH]3)c3cc(F)ccc3O)C4=O)cc2)CC1] |
| 007 | 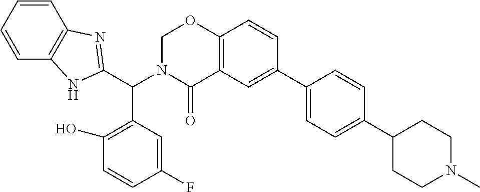 [SMILES: CN1CCC(c2ccc(-c3ccc4c(c3)C(=O)N(C(c3nc5ccccc5[nH]3)c3cc(F)ccc3O)CO4)cc2)CC1] |

or a pharmaceutically acceptable salt thereof.

In another aspect, provided herein is a compound of Formula III:

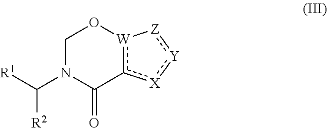

or a pharmaceutically acceptable salt thereof;
   wherein
[symbol] represents an optional double bond;
W is N, C, or CH;
Z is selected from the group consisting of S, O, N, NH, CH₂, C-halo, and CH;
X and Y are each, independently, S, O, N, CH, NR³, or CR³;
provided that at least one of X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl, all of which are optionally substituted with one, two, or three R⁸;
R² is selected from the group consisting of 6-10 membered aryl, 5-10 membered heteroaryl, 3-10 membered heterocycloalkyl, and 3-10 membered cycloalkyl, all of which are optionally substituted with one, two, or three R⁶;
R³ is independently, at each occurrence, selected from the group consisting of halogen, OR⁴, NR⁴R⁴, SO₂R⁴, SO₂NHR⁴, NHSO₂R⁴, C(O)OR⁴, C(O)NHR⁴, C(O)R⁴, C₁-C₆ alkyl, C₂-C₆ alkenyl, C₂-C₆ alkynyl, 3-7 membered cycloalkyl, C₄-C₇ cycloalkenyl, C₆-C₁₀ aryl, 5-6 membered heteroaryl, and 5-7 membered heterocyclyl, wherein alkyl, alkenyl, or alkynyl are each optionally substituted one, two, or three times with R⁴, and wherein aryl, heteroaryl, or heterocyclyl are each optionally substituted one, two, or three times with R⁵;
R⁴ is independently, at each occurrence, selected from the group consisting of H, (CH₂)₀₋₃—(C₃-C₇ cycloalkyl), (CH₂)₀₋₃—(C₄-C₇ cycloalkenyl), (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁵;
R⁵ is independently, at each occurrence, selected from the group consisting of C₁-C₆ alkyl, C₁-C₆ haloalkyl, C₁-C₆ alkoxy, C₁-C₆ haloalkoxy, C₁-C₃ alkylamine, 3-10 membered cycloalkyl, halogen, COOH, C(O)O(C₁-C₆ alkyl), O(CH₂)₁₋₃—OH, NH₂, NH(C₁-C₆ alkyl), N(C₁-C₆ alkyl)₂, OH, CN, (CH₂)₀₋₃—(C₆-C₁₀ aryl), (CH₂)₀₋₃-(5-6 membered heteroaryl), O(CH₂)₀₋₃-(4-7 membered heterocyclyl), and (CH₂)₀₋₃-(4-7 membered heterocyclyl), wherein the alkyl, alkoxy, aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN;
alternatively, two R⁶, together with the atoms to which they are attached, can form 5-10 membered heteroaryl, 6-10 membered aryl, 3-10 membered heterocycloalkyl, or 3-10 membered cycloalkyl;
R⁷ is independently, at each occurrence, selected from the group consisting of substituents independently selected from C₁-C₆ alkyl, C₁-C₆ haloalkyl, C₁-C₆ alkoxy, C₁-C₆ haloalkoxy, halogen, NH₂, NH(C₁-C₆ alkyl), N(C₁-C₆ alkyl)₂, SO₂NH₂, SO₂NH(C₁-C₆ alkyl), SO₂N(C₁-C₆ alkyl)₂, (CH₂)₁₋₂—OH, C(O)(CH₂)₁₋₂—OH, C(O)(C₁-C₆ alkyl), and C(O)O(C₁-C₆ alkyl);
alternatively, two R⁷, together with the atoms to which they are attached, can form 5-10 membered heteroaryl, 6-10 membered aryl, 3-10 membered heterocycloalkyl, or 3-10 membered cycloalkyl;
R⁸ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, 3-6 membered cycloalkyl, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN; and
R⁹ is selected from the group consisting of 6-10 membered aryl, 5-10 membered heteroaryl, 3-10 membered heterocycloalkyl, and 3-10 membered cycloalkyl.

In an embodiment of Formula III, or a pharmaceutically acceptable salt thereof,
[symbol] represents an optional double bond;
W is C;
Z is selected from the group consisting of S, O, N, NH, CH₂, C-halo, and CH;
X and Y are each, independently, S, O, N, CH, NR³, or CR³;
provided that at least one of X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl;
R² is selected from the group consisting of 6-10 membered aryl and 5-10 membered heteroaryl, both of which are optionally substituted with one, two, or three R⁶;
R³ is independently, at each occurrence, selected from the group consisting of C₆-C₁₀ aryl, 5-6 membered heteroaryl, and 5-7 membered heterocyclyl, wherein aryl, heteroaryl, or heterocyclyl are each optionally substituted one, two, or three times with R⁵;
R⁵ is independently, at each occurrence, selected from the group consisting of 3-10 membered cycloalkyl, (CH₂)₀₋₃—(C₆-C₁₀ aryl), (CH₂)₀₋₃-(5-6 membered heteroaryl), O(CH₂)₀₋₃-(4-7 membered heterocyclyl), and (CH₂)₀₋₃-(4-7 membered heterocyclyl), wherein the aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, and OH;
R⁷ is independently, at each occurrence, selected from the group consisting of C₁-C₆ alkyl, C₁-C₆ haloalkyl, C₁-C₆ alkoxy, and halogen;
R⁸ is selected from the group consisting of hydrogen, C₁-C₃ alkyl, C₁-C₃ haloalkyl, and C₁-C₃ alkoxy; and
R⁹ is 5-10 membered heteroaryl.

In an embodiment, at least one of X, Y, or Z is S. In another embodiment, no more than two X, Y, or Z is S or N. In yet another embodiment, no more than one X, Y, or Z is S or N. In yet another embodiment, only one of X or Y is CR³. In still another embodiment, X is CR³. In another embodiment, Y is CR³.

In another embodiment, the compound of Formula III is a compound of Formula Illa:

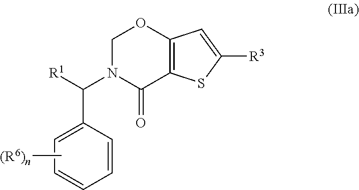

or a pharmaceutically acceptable salt thereof;
wherein n is 0, 1, or 2.

In another embodiment, the compound of Formula III is a compound of Formula IIIb:

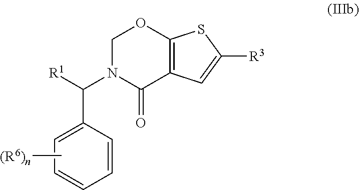

or a pharmaceutically acceptable salt thereof;
wherein n is 0, 1, or 2.

In an embodiment, n is 1. In another embodiment, n is 1. In yet another embodiment, nis 2.

In yet another embodiment, wherein R¹ is C(O)NHR⁹ or 5-10 membered heteroaryl.

In still another embodiment, R¹ is selected from the group consisting of:

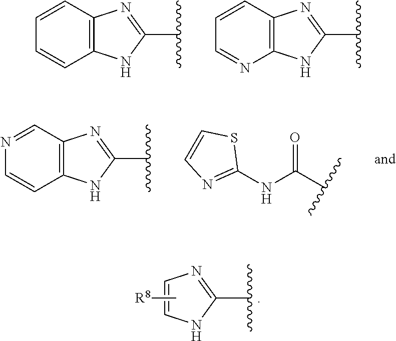

In an embodiment, wherein R⁸ is C₁-C₃ alkyl.

In another embodiment, R¹ is

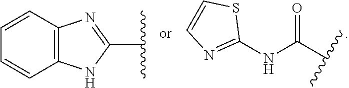

In yet another embodiment,
R³ is independently, at each occurrence, C₆-C₁₀ aryl optionally substituted one, two, or three times with R⁵;
R⁵ is independently, at each occurrence, selected from the group consisting of (CH₂)₀₋₃-(5-7 membered heterocyclyl) optionally substituted one, two, or three times with R⁷; and
R⁷ is independently, at each occurrence, selected from the group consisting of C₁-C₆ alkyl.

In still another embodiment, R³ is independently, at each occurrence, selected from the group consisting of

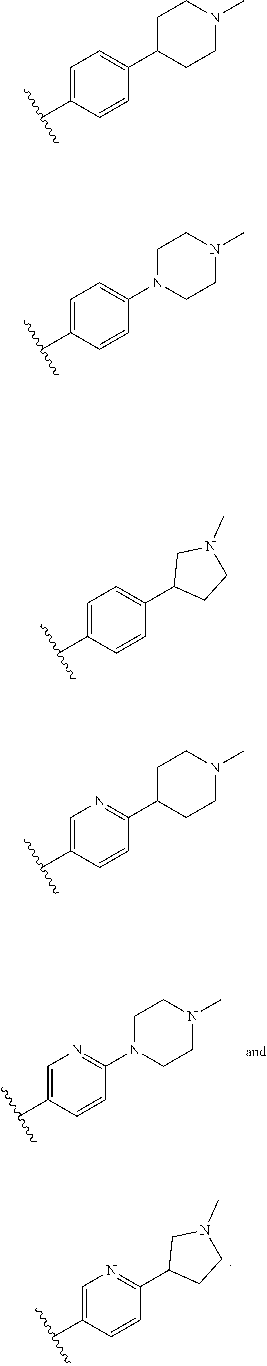 [SMILES: *c1ccc(C2CCN(C)CC2)cc1 ; *c1ccc(N2CCN(C)CC2)cc1 ; *c1ccc(C2CCN(C)C2)cc1 ; *c1ccc(C2CCN(C)CC2)nc1 ; *c1ccc(N2CCN(C)CC2)nc1 ; *c1ccc(C2CCN(C)C2)nc1]

In an embodiment, R³ is independently, at each occurrence, selected from the group consisting of

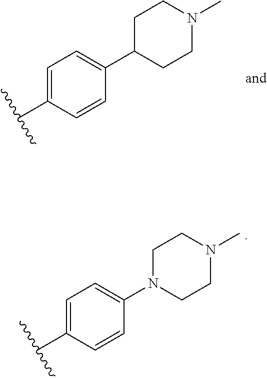 [SMILES: *c1ccc(C2CCN(C)CC2)cc1 ; *c1ccc(N2CCN(C)CC2)cc1]

In another embodiment, the compound of Formula III is a compound of Formula Illa, or a pharmaceutically acceptable salt thereof;
wherein
R¹ is selected from the group consisting of C(O)NHR⁹,

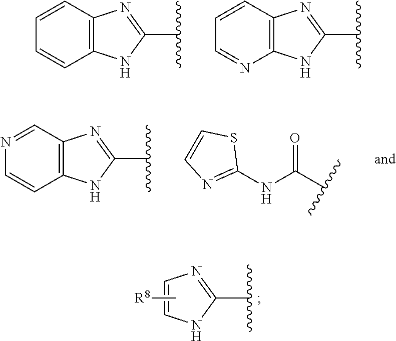

R⁸ is C₁-C₃ alkyl;
R³ is selected from the group consisting of

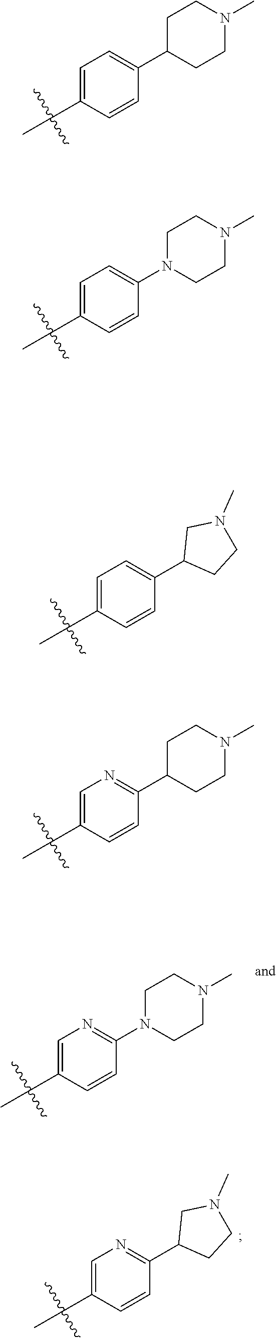 [SMILES: *c1ccc(C2CCN(C)CC2)cc1 ; *c1ccc(N2CCN(C)CC2)cc1 ; *c1ccc(C2CCN(C)C2)cc1 ; *c1ccc(C2CCN(C)CC2)nc1 ; *c1ccc(N2CCN(C)CC2)nc1 ; *c1ccc(C2CCN(C)C2)nc1]

and
R⁹ is 5-membered heteroaryl.

In another embodiment, the compound of Formula III is selected from the group consisting of a compound in Table 2.

TABLE 2

| Compound No. | Structure |
|---|---|
| 008 | 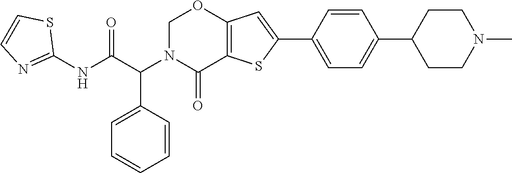 [SMILES: CN1CCC(c2ccc(-c3cc4c(s3)C(=O)N(C(C(=O)Nc3nccs3)c3ccccc3)CO4)cc2)CC1] |
| 009 | 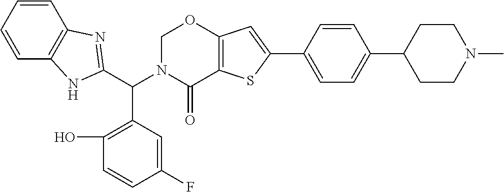 [SMILES: CN1CCC(c2ccc(-c3cc4c(s3)C(=O)N(C(c3nc5ccccc5[nH]3)c3cc(F)ccc3O)CO4)cc2)CC1] |
| 010 | 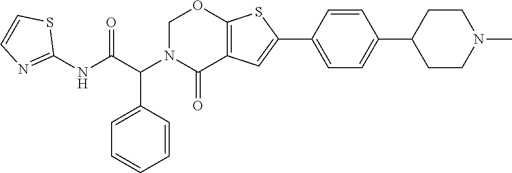 [SMILES: CN1CCC(c2ccc(-c3cc4c(s3)OCN(C(C(=O)Nc3nccs3)c3ccccc3)C4=O)cc2)CC1] |

or a pharmaceutically acceptable salt thereof.

The compounds disclosed herein may exist as tautomers and optical isomers (e.g., enantiomers, diastereomers, diastereomeric mixtures, racemic mixtures, and the like).

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

It is generally well known in the art that any compound that will be converted in vivo to provide a compound disclosed herein is a prodrug within the scope of the present disclosure.

In an aspect, provided herein is a pharmaceutical composition comprising any one of the compounds disclosed herein, or a pharmaceutically acceptable salt thereof, and at least one pharmaceutically acceptable carrier.

In an embodiment, the composition further comprises a second active agent. In another embodiment, the second active agent is selected from the group consisting of a MEK inhibitor, a PI3K inhibitor, and an mTor inhibitor. In yet another embodiment, the second active agent prevents EGFR dimer formation in a subject. In still another embodiment, the second active agent is selected from the group consisting of cetuximab, trastuzumab, and panitumumab. In an embodiment, the second active agent is an ATP competitive EGFR inhibitor. In another embodiment, the ATP competitive EGFR inhibitor is osimertinib, gefitinib, or erlotinib. In another embodiment, the ATP competitive EGFR inhibitor is osimertinib.

In another aspect, provided herein are pharmaceutical compositions comprising a compound of the present disclosure, or a pharmaceutically acceptable salt thereof, and a pharmaceutically acceptable carrier. In another aspect, the pharmaceutical composition further comprises a second active agent, wherein said second active agent prevents EGFR dimer formation, and a pharmaceutically acceptable carrier. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

A compound that binds to an allosteric site in EGFR, such as the compounds of the present disclosure (e.g., the compounds of the formulae disclosed herein), optionally in combination with a second active agent, wherein said second active agent prevents EGFR dimer formation, are capable of modulating EGFR activity. In some embodiments, the compounds of the present disclosure are capable of inhibiting or decreasing EGFR activity without a second active agent (e.g., an antibody such as cetuximab, trastuzumab, or panitumumab). In other embodiments, the compounds of the present disclosure in combination with a second active agent. In an embodiment, the second active agent prevents EGFR dimer formation and/or are capable of inhibiting or decreasing EGFR activity. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

### Methods of Treatment

In an aspect, provided herein is a method of treating cancer in an individual in need thereof, comprising administering to the individual a therapeutically effective amount of a compound disclosed herein. In an embodiment, the cancer is selected from the group consisting of lung cancer, colon cancer, breast cancer, endometrial cancer, thyroid cancer, glioma, squamous cell carcinoma, and prostate cancer. In another embodiment, the cancer is non-small cell lung cancer (NSCLC).

In another aspect, provided herein is a method of inhibiting a kinase in an individual in need thereof, comprising administering to the individual a therapeutically effective amount of a compound provided herein. In an embodiment, the kinase is EGFR.

In yet another aspect, provided herein is a method of treating or preventing a kinase-mediated disorder in an individual in need thereof, comprising administering to the individual a therapeutically effective amount of a compound of the present disclosure. In an embodiment, the kinase-mediated disorder is resistant to an EGFR-targeted therapy. In another embodiment, the EGFR-treated therapy is selected from the group consisting of gefitinib, erlotinib, or osimertinib.

In some embodiments, the compounds of the present disclosure are capable of modulating (e.g., inhibiting or decreasing) the activity of EGFR containing one or more mutations. In some embodiments, the mutant EGFR contains one or more mutations selected from T790M, L718Q, L844V, V948R, L858R, 1941R, and C797S. In other embodiments, the mutant EGFR contains a combination of mutations, wherein the combination is selected from L858R/L718Q, L858R/L844V, L858R/T790M, L858R/T790M/1941R, L858R/C797S, L858R/T790M/C797S, and L858R/T790M/L718Q. In other embodiments, the mutant EGFR contains a combination of mutations, wherein the combination is selected from L858R/L844V, L858R/T790M, L858R/T790M/1941R, L858R/C797S, L858R/T790M/C797S, and L858R/T790M. In other embodiments, the mutant EGFR contains a combination of mutations, wherein the combination is selected from L858R/T790M, L858R/T790M/1941R, L858R/C797S, L858R/T790M/C797S, and L858R/T790M.

In some embodiments, the compounds of the present disclosure in combination with a second active agent, wherein said second active agent prevents EGFR dimer formation, are capable of modulating (e.g., inhibiting or decreasing) the activity of EGFR containing one or more mutations. In some embodiments, the mutant EGFR contains one or more mutations selected from T790M, L718Q, L844V, V948R, L858R, 1941R, and C797S. In other embodiments, the mutant EGFR contains a combination of mutations, wherein the combination is selected from L858R/L718Q, L858R/L844V, L858R/T790M, L858R/T790M/1941R, L858R/C797S, L858R/T790M/C797S, and L858R/T790M/L718Q. In other embodiments, the mutant EGFR contains a combination of mutations, wherein the combination is selected from L858R/L844V, L858R/T790M, L858R/T790M/1941R, L858R/C797S, L858R/T790M/C797S, and L858R/T790M. In other embodiments, the mutant EGFR contains a combination of mutations, wherein the combination is selected from L858R/T790M, L858R/T790M/1941R, L858R/C797S, L858R/T790M/C797S, and L858R/T790M. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib.

In some embodiments, the compounds of the present disclosure are capable of modulating (e.g., inhibiting or decreasing) the activity of EGFR containing one or more mutations, but do not affect the activity of a wild-type EGFR.

In other embodiments, the compounds of the present disclosure in combination with a second active agent, wherein said second active agent prevents EGFR dimer formation, are capable of modulating (e.g., inhibiting or decreasing) the activity of EGFR containing one or more mutations, but do not affect the activity of a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

Modulation of EGFR containing one or more mutations, such as those described herein, but not a wild-type EGFR, provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the inhibition of EGFR activity is measured by IC₅₀.

In some embodiments, the inhibition of EGFR activity is measured by EC₅₀.

In some embodiments, the inhibition of EGFR by a compound of the disclosure can be measured via a biochemical assay. By illustrative and non-limiting example, a homogenous time-resolved fluorescence (HTRF) assay may be used to determine inhibition of EGFR activity using conditions and experimental parameters disclosed herein. The HTRF assay may, for example, employ concentrations of substrate (e.g., biotin-Lck-peptide substrate) of about 1 μM; concentrations of EGFR (mutant or WT) from about 0.2 nM to about 40 nM; and concentrations of inhibitor from about 0.000282 μM to about 50 μM. A compound of the disclosure screened under these conditions may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM. In certain embodiments, a compound of the disclosure screened under the above conditions for inhibition of EGFR having a mutation or combination of mutations selected from L858R/T790M, L858R, and T790M may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

In some embodiments, the compounds of the disclosure bind to an allosteric site in EGFR. In some embodiments, the compounds of the disclosure interact with at least one amino acid residue of epidermal growth factor receptor (EGFR) selected from Lys745, Leu788, and Ala 743. In other embodiments, the compounds of the disclosure interact with at least one amino acid residue of epidermal growth factor receptor (EGFR) selected from Cys755, Leu777, Phe856, and Asp855. In other embodiments, the compounds of the disclosure interact with at least one amino acid residue of epidermal growth factor receptor (EGFR) selected from Met766, Ile759, Glu762, and Ala763. In other embodiments, the compounds of the disclosure interact with at least one amino acid residue of epidermal growth factor receptor (EGFR) selected from Lys745, Leu788, and Ala 743; at least one amino acid residue of epidermal growth factor receptor (EGFR) selected from Cys755, Leu777, Phe856, and Asp855; and at least one amino acid residue of epidermal growth factor receptor (EGFR) selected from Met766, Ile759, Glu762, and Ala763. In other embodiments, the compounds of the disclosure do not interact with any of the amino acid residues of epidermal growth factor receptor (EGFR) selected from Met793, Gly796, and Cys797.

In some embodiments, the disclosure provides a compound comprising an allosteric kinase inhibitor, wherein the compound is a more potent inhibitor of a drug-resistant EGFR mutant relative to a wild type EGFR. In some embodiments, the drug-resistant EGFR mutant is resistant to one or more known EGFR inhibitors, including but not limited to gefitinib, erlotinib, lapatinib, HKI-272, and osimertinib.

In some embodiments, the drug-resistant EGFR mutant comprises a sensitizing mutation, such as L858R.

In some embodiments, the disclosure provides a compound comprising an allosteric kinase inhibitor in combination with a second active agent, wherein said second active agent prevents EGFR dimer formation, wherein the compound is a more potent inhibitor of a drug-resistant EGFR mutant relative to a wild type EGFR. In some embodiments, the drug-resistant EGFR mutant is resistant to one or more known EGFR inhibitors, including but not limited to gefitinib, erlotinib, lapatinib, HKI-272, and osimertinib. In some embodiments, the drug-resistant EGFR mutant comprises a sensitizing mutation, such as L858R. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib.

In other embodiments, the disclosure provides a compound comprising an allosteric kinase inhibitor in combination with a second active agent, wherein said second active agent prevents EGFR dimer formation, wherein the compound in combination with the second active agent is more potent than one or more known EGFR inhibitors, including but not limited to gefitinib, erlotinib, lapatinib, HKI-272, and osimertinib, at inhibiting the activity of EGFR containing one or more mutations as described herein, such as T790M, L718Q, L844V, L858R, and C797S. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In other embodiments, the disclosure provides a compound comprising an allosteric kinase inhibitor in combination with a second active agent, wherein said second active agent prevents EGFR dimer formation, wherein the compound in combination with the second active agent is less potent than one or more known EGFR inhibitors, including but not limited to gefitinib, erlotinib, lapatinib, HKI-272, and osimertinib, at inhibiting the activity of a wild-type EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

Potency of the inhibitor can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

Potency of the inhibitor can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

An EGFR sensitizing mutation comprises without limitation L858R, G719S, G719C, G719A, and/or L861Q. A drug-resistant EGFR mutant can have without limitation a drug resistance mutation comprising T790M, T854A, L718Q, C797S, or D761Y.

The selectivity between wild-type EGFR and EGFR containing one or more mutations as described herein can also be measured using cellular proliferation assays where cell proliferation is dependent on kinase activity. For example, murine Ba/F3 cells transfected with a suitable version of wild-type EGFR (such as VIII; containing a WT EGFR kinase domain), or Ba/F3 cells transfected with L858R/T790M, L858R/T790M/L718Q, L858R/C797S, L858R/T790M/C797S, or L858R/T790M/1941R can be used. Proliferation assays are performed at a range of inhibitor concentrations (10 μM, 3 μM, 1.1 μM, 330 nM, 110 nM, 33 nM, 11 nM, 3 nM, I nM) and an EC₅₀ is calculated.

An alternative method to measure effects on EGFR activity is to assay EGFR phosphorylation. Wild type or mutant (L858R/T790M, L858R/C797S, L858R/T790M/C797S, L858R/T790M/1941R, or L858R/T790M/L718Q) EGFR can be transfected into NIH-3T3 cells (which do not normally express endogenous EGFR) and the ability of the inhibitor (using concentrations as above) to inhibit EGFR phosphorylation can be assayed. Cells are exposed to increasing concentrations of inhibitor for 6 hours and stimulated with EGF for 10 minutes. The effects on EGFR phosphorylation are assayed by Western Blotting using phospho-specific (Y1068) EGFR antibodies.

In still another aspect, the disclosure provides a method of inhibiting epidermal growth factor receptor (EGFR), the method comprising administering to a subject in need thereof an effective amount of a compound of disclosed herein, or a pharmaceutically acceptable salt thereof. In some embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In another aspect, provided herein is a method of treating or preventing a disease, the method comprising administering to a subject in need thereof an effective amount of a compound of disclosed herein, or a pharmaceutically acceptable salt thereof. In some embodiments, the disease is mediated by a kinase. In further embodiments, the kinase comprises a mutated cysteine residue. In further embodiments, the mutated cysteine residue is located in or near the position equivalent to Cys 797 in EGFR, including such positions in Jak3, Blk, Bmx, Btk, HER2 (ErbB2), HER4 (ErbB4), Itk, Tec, and Txk. In some embodiments, the method further comprises administering a second active agent, wherein said second active agent prevents dimer formation of the kinase. In some embodiments, the second active agent that prevents kinase dimer formation is an antibody. In further embodiments, the second active agent prevents EGFR dimer formation. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In some embodiments, the disease is mediated by EGFR (e.g., EGFR plays a role in the initiation or development of the disease). In some embodiments, the disease is mediated by a Her-kinase. In further embodiments, the Her-kinase is HER1, HER2, or HER4.

In certain embodiments, the disease is resistant to a known EGFR inhibitor, including but not limited to, gefitinib, erlotinib, or osimertinib. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, and/or L861Q. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In certain embodiments, the disease is cancer or a proliferation disease.

In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In certain embodiments, the disease is resistant to a known EGFR inhibitor, including but not limited to, gefitinib, erlotinib, or osimertinib. In certain embodiments, a diagnostic test is performed to determine if the disease is associated with an activating mutation in EGFR. In certain embodiments, a diagnostic test is performed to determine if the disease is associated with an EGFR harboring an activating mutation and/or a drug resistance mutation. Activating mutations comprise without limitation L858R, G719S, G719C, G719A, L718Q, and/or L861Q. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In yet another aspect, provided herein is a method of treating a kinase-mediated disorder comprising administering to a subject in need thereof an effective amount of a compound disclosed herein, or a pharmaceutically acceptable salt thereof. In some embodiments, the compound is an inhibitor of HER1, HER2, or HER4. In other embodiments, the subject is administered an additional therapeutic agent. In other embodiments, the compound and the additional therapeutic agent are administered simultaneously or sequentially.

In another aspect, the disclosure provides a method of treating a kinase mediated disorder, the method comprising administering to a subject in need thereof an effective amount of a compound of disclosed herein, or a pharmaceutically acceptable salt thereof, and a second active agent, wherein said second active agent prevents EGFR dimer formation. In some embodiments, the compound is an inhibitor of HER1, HER2, or HER4. In other embodiments, the subject is administered an additional therapeutic agent. In other embodiments, the compound, the second active agent that prevents EGFR dimer formation, and the additional therapeutic agent are administered simultaneously or sequentially. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In other embodiments, the disease is cancer. In further embodiments, the cancer is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors. In further embodiments, the disease is lung cancer, breast cancer, glioma, squamous cell carcinoma, or prostate cancer. In still further embodiments, the disease is non-small cell lung cancer.

In another aspect, provided herein is a method of treating cancer, wherein the cancer cell comprises activated EGFR, comprising administering to a subject in need thereof an effective amount of a compound of disclosed herein, or a pharmaceutically acceptable salt thereof.

In another aspect, provided herein is a method of treating cancer, wherein the cancer cell comprises activated EGFR, comprising administering to a subject in need thereof an effective amount of a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

In certain embodiments, the EGFR activation is selected from mutation of EGFR, amplification of EGFR, expression of EGFR, and ligand mediated activation of EGFR.

In further embodiments, the mutation of EGFR is selected from G719S, G719C, G719A, L858R, and L861Q.

In still another aspect, provided herein is a method of treating cancer in a subject, wherein the subject is identified as being in need of EGFR inhibition for the treatment of cancer, comprising administering to the subject an effective amount of a compound disclosed herein, or a pharmaceutically acceptable salt thereof.

In certain embodiments, the subject identified as being in need of EGFR inhibition is resistant to a known EGFR inhibitor, including but not limited to, gefitinib, erlotinib, or osimertinib. In certain embodiments, a diagnostic test is performed to determine if the subject has an activating mutation in EGFR. In certain embodiments, a diagnostic test is performed to determine if the subject has an EGFR harboring an activating mutation and/or a drug resistance mutation. Activating mutations comprise without limitation L858R, G719S, G719C, G719A, L718Q, and/or L861Q. Drug resistant EGFR mutants can have without limitation a drug resistance mutation comprising T790M, T854A, L718Q, C797S, or D761Y. The diagnostic test can comprise sequencing, pyrosequencing, PCR, RT-PCR, or similar analysis techniques known to those of skill in the art that can detect nucleotide sequences.

In an aspect, provided herein is a method of preventing resistance to a known EGFR inhibitor (including but not limited to gefitinib, erlotinib, or osimertinib) in a subject, comprising administering to a subject in need thereof an effective amount of a compound disclosed herein, or a pharmaceutically acceptable salt thereof.

In another aspect, provided herein is a method of preventing resistance to a known EGFR inhibitor (including but not limited to gefitinib, erlotinib, or osimertinib) in a disease, comprising administering to a subject in need thereof an effective amount of a compound disclosed herein, or a pharmaceutically acceptable salt thereof, and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In an embodiment of the methods disclosed herein, the subject is a human.

In another aspect, the disclosure provides a compound disclosed herein, or a pharmaceutically acceptable salt thereof, for use in the manufacture of a medicament for treating or preventing a disease in which EGFR plays a role.

In an aspect, provided herein is a method of treating or preventing a condition selected from the group consisting of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, said condition is selected from a proliferative disorder and a neurodegenerative disorder.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia. The term “cancer” includes, but is not limited to, the following cancers: myeloma, lymphoma, or a cancer selected from gastric, renal, head and neck, oropharangeal, non-small cell lung cancer (NSCLC), endometrial, hepatocarcinoma, non-Hodgkin's lymphoma, and pulmonary.

The term “cancer” also refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the disclosure, the present disclosure provides for the use of one or more compounds of the disclosure in the manufacture of a medicament for the treatment of cancer, including without limitation the various types of cancer disclosed herein.

In some embodiments, the compounds of this disclosure are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds of this disclosure are useful for treating hematopoietic disorders, in particular, acute-myelogenous leukemia (AML), chronic-myelogenous leukemia (CML), acute-promyelocytic leukemia, and acute lymphocytic leukemia (ALL).

The term “cancerous cell” as provided herein, includes a cell afflicted by any one of the above-identified conditions.

The disclosure further provides a method for the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof. In other embodiments, the method further comprises administering a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The activity of the compounds and compositions of the present disclosure as EGFR kinase inhibitors may be assayed in vitro, in vivo, or in a cell line. In vitro assays include assays that determine inhibition of either the kinase activity or ATPase activity of the activated kinase. Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound utilized in this disclosure as an inhibitor of various kinases are set forth in the Examples below.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent, wherein said second active agent prevents EGFR dimer formation. For any of the above uses, the required dosage will vary depending on the mode of administration, the particular condition to be treated and the effect desired.

In other embodiments, the compound and the second active agent that prevents EGFR dimer formation are administered simultaneously or sequentially.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/Kg to about 500 mg/Kg, alternatively from about 1 to about 50 mg/Kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, an agent that prevents EGFR dimer formation, chemotherapeutic agents or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat; sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present disclosure.

### Kits

In an aspect, provided herein is a kit comprising a compound capable of inhibiting kinase activity selected from one or more compounds of disclosed herein, or pharmaceutically acceptable salts thereof, and instructions for use in treating cancer. In certain embodiments, the kit further comprises components for performing a test to determine whether a subject has activating and/or drug resistance mutations in EGFR.

In another aspect, the disclosure provides a kit comprising a compound capable of inhibiting EGFR activity selected from a compound disclosed herein, or a pharmaceutically acceptable salt thereof.

In another aspect, the disclosure provides a kit comprising a compound capable of inhibiting kinase activity selected from one or more compounds of disclosed herein, or pharmaceutically acceptable salts thereof; a second active agent, wherein said second active agent prevents EGFR dimer formation; and instructions for use in treating cancer. In certain embodiments, the kit further comprises components for performing a test to determine whether a subject has activating and/or drug resistance mutations in EGFR. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab.

In another aspect, the disclosure provides a kit comprising a compound capable of inhibiting EGFR activity selected from a compound of disclosed herein, or a pharmaceutically acceptable salt thereof and a second active agent, wherein said second active agent prevents EGFR dimer formation. In some embodiments, the second active agent that prevents EGFR dimer formation is an antibody. In further embodiments, the second active agent that prevents EGFR dimer formation is cetuximab, trastuzumab, or panitumumab. In further embodiments, the second active agent that prevents EGFR dimer formation is cetuximab. In an embodiment, the second active agent is an ATP competitive EGFR inhibitor. In another embodiment, the ATP competitive EGFR inhibitor is osimertinib, gefitinib or erlotinib. In another embodiment, the ATP competitive EGFR inhibitor is osimertinib.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Abbreviations

AcOH acetic acid
ACN acetonitrile
DCM dichloromethane
DIEA diispropylethylamine
DMF dimethylformamide
EtOAc ethyl acetate
HATU (1-[bis(dimethylamino)methylene]-1H-1,2,3-triazolo[4,5-b]pyridinium 3-oxide hexafluorophosphate
MeOH methanol
RT room temperature
TFA trifluoroacetic acid
THF tetrahydrofuran
tol toluene
TsOH p-toluenesulfonic acid

### Example 1: Synthetic Procedures

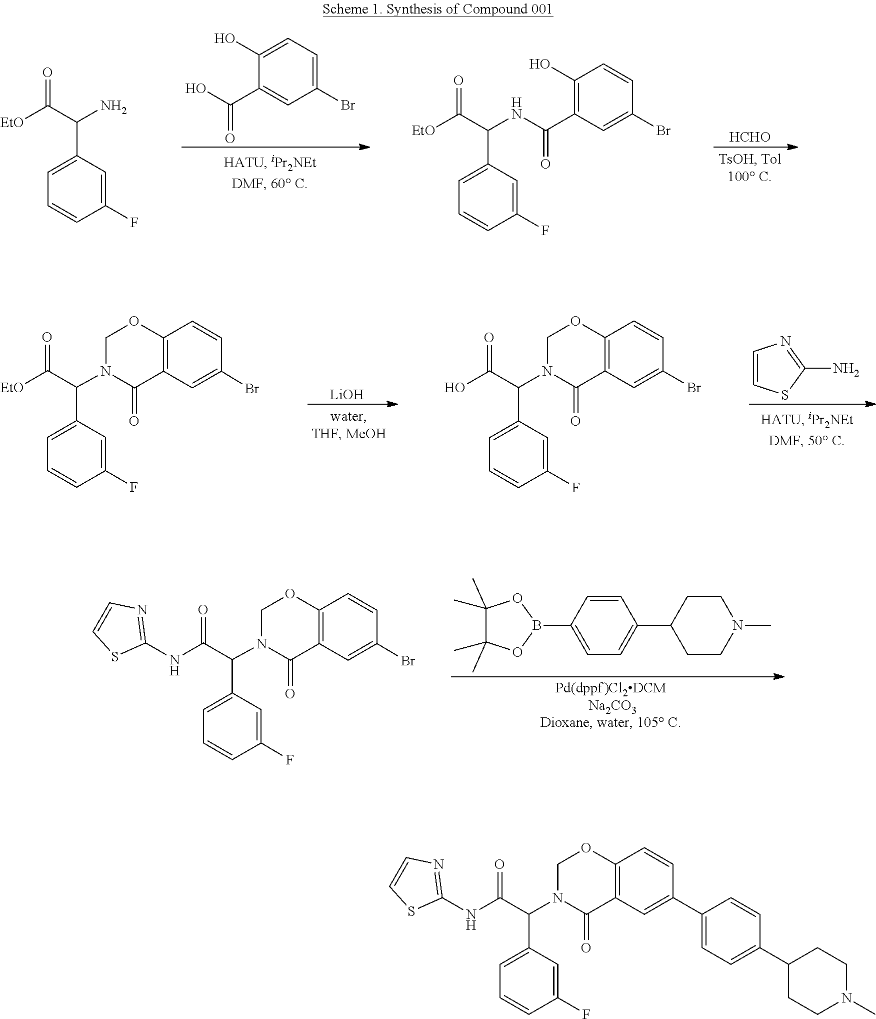

### Ethyl 2-(5-bromo-2-hydroxybenzamido)-2-(3-fluorophenyl)acetate

Diisopropylethylamine (870 uL, 5 mmol) was added to a mixture of ethyl 2-amino-2-(3-fluorophenyl)acetate (350 mg, 1.5 mmol), 5-bromo-2-hydroxybenzoic acid (217 mg, 1 mmol) and HATU (760 mg, 2 mmol) in DMF (3 mL). The reaction mixture was stirred at 60° C. for 1.5 hours, allowed to cool, poured into brine (20 mL) and extracted with of EtOAc (3×20 mL). The combined organic extracts were washed with water and brine, dried over Na₂SO₄, filtered, and concentrated under reduced pressure. The residue was purified by flash chromatography (0-80% EtOAc in hexane) to give the title compound (261 mg, 66%). ¹H NMR (500 MHZ, CDCl₃) δ: 11.85 (s, 1H) 7.63 (d, 1H) 7.52 (dd, 1H) 7.39 (m, 2H) 7.25 (m, 1H), 7.17 (m, 1H) 7.08 (m, 1H) 6.90 (d, 1H) 5.70 (d, 1H) 4.33 (q, 1H) 4.26 (q, 1H) 1.28 (t, 3H); MS m/z: [M+1]⁺, 396.0.

### Ethyl 2-(6-bromo-4-oxo-2H-benzo[e][1,3]oxazin-3 (4H)-yl)-2-(3-fluorophenyl)acetate

A mixture of ethyl 2-(5-bromo-2-hydroxybenzamido)-2-(3-fluorophenyl)acetate (261 mg, 0.66 mmol), paraformaldehyde (42 mg, 1.45 mmol), 4-methylbenzenesulfonic acid (250 mg, 1.39 mmol) in toluene (5 mL) was heated to 100° C. for 2 hours. The solvent was removed under reduced pressure. The residue was purified by flash chromatography (0-30% EtOAc in hexane) to give the title compound (250 mg, 93%). ¹H NMR (500 MHz, CDCl₃) δ: 8.14 (d, 1H) 7.56 (dd, 1H) 7.42 (m, 1H) 7.13 (m, 2H) 7.08 (m, 1H) 6.87 (d, 1H) 6.51 (s, 1H) 5.47 (d, 1H) 5.01 (d, 1H) 4.35 (m, 2H) 1.35 (t, 3H); MS m/z: [M+1]⁺, 408.0.

### 2-(6-Bromo-4-oxo-2H-benzo[e][1,3]oxazin-3 (4H)-yl)-2-(3-fluorophenyl)acetic acid

A mixture of ethyl 2-(6-bromo-4-oxo-2H-benzo[e][1,3]oxazin-3 (4H)-yl)-2-(3-fluorophenyl)acetate (250 mg, 0.61 mmol) in water (2 mL), THF (2 mL), and MeOH (2 mL) was treated with LiOH—H₂O (64 mg, 1.53 mmol). The reaction mixture was stirred for 15 min at RT. The solvent was removed under reduced pressure and the residue was dissolved in water (15 mL). The solution was acidified to pH 3 with 6 N HCl and extracted with EtOAc (3×20 mL). The combined organic extracts were dried over Na₂SO₄, filtered, and concentrated under reduced pressure to give the title compound, (225 mg, 97%). ¹H NMR (500 MHZ, CDCl₃) δ: 8.12 (d, 1H) 7.57 (dd, 1H) 7.44 (m, 1H) 7.20 (d, 1H) 7.14 (m, 2H) 6.86 (d, 1H) 6.52 (s, 1H) 5.41 (d, 1H) 4.99 (d, 1H); MS m/z: [M+1]⁺, 379.9.

### 2-(6-Bromo-4-oxo-2H-benzo[e][1,3]oxazin-3 (4H)-yl)-2-(3-fluorophenyl)-N-(thiazol-2-yl)acetamide

Diisopropylethylamine (177 uL, 1.02 mmol) was added to a mixture of 2-(6-bromo-4-oxo-2H-benzo[e][1,3]oxazin-3 (4H)-yl)-2-(3-fluorophenyl)acetic acid (130 mg, 0.34 mmol), 2-aminothiazole (51 mg, 0.51 mmol) and HATU (258 mg, 0.68 mmol) in DMF (1.5 mL). The reaction mixture was stirred at 50° C. for 1.5 hours. After cooling to RT, the mixture was poured into water (20 mL), and the resulting precipitate was filtered, washed with water, and dried to give the title compound (95 mg, 60%). ¹H NMR (500 MHZ, DMSO-d₆) δ: 7.94 (d, 1H) 7.74 (dd, 1H) 7.52 (m, 2H) 7.33 (m, 2H) 7.17 (m, 2H) 7.08 (d, 1H) 6.53 (s, 1H) 5.48 (d, 1H) 5.12 (d, 1H); MS m/z: [M+1]⁺, 461.9.

### Compound 1 2-(3-Fluorophenyl)-2-(6-(4-(1-methylpiperidin-4-yl)phenyl)-4-oxo-2H-benzo[e][1,3]oxazin-3 (4H)-yl)-N-(thiazol-2-yl)acetamide

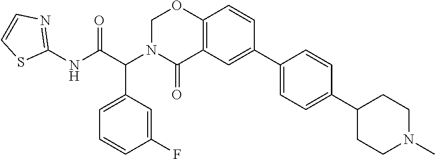 [SMILES: CN1CCC(c2ccc(-c3ccc4c(c3)C(=O)N(C(C(=O)Nc3nccs3)c3cccc(F)c3)CO4)cc2)CC1]

A mixture of 2-(6-bromo-4-oxo-2H-benzo[e][1,3]oxazin-3 (4H)-yl)-2-(3-fluorophenyl)-N-(thiazol-2-yl)acetamide (45 mg, 0.097 mmol), Na₂CO₃ (31 mg, 0.29 mmol) and 1-methyl-4-(4-(4,4,5,5-tetramethyl-1,3,2-dioxaborolan-2-yl)phenyl) piperidine (36 mg, 0.12 mmol) in dioxane (2 mL) and water (0.5 mL) in a sealed tube was degassed and suffused with N₂ three times. [1,1′-Bis(diphenylphosphino) ferrocene]dichloropalladium (II) (15 mg, 0.009 mmol) was added and the vessel was resealed and suffused with N₂. The mixture was heated to 105° C. for 1 hour. After cooling, the entire reaction mixture was purified by reverse phase-HPLC, eluting with 0-80%, ACN/H₂O (0.038% TFA modifier) to give the title compound (10 mg, 19%). ¹H NMR (500 MHZ, DMSO-d₆) δ: 9.29 (br s, 1H) 8.08 (d, 1H) 7.88 (dd, 1H) 7.65 (d, 2H) 7.54 (m, 1H) 7.53 (d, 1H) 7.35 (d, 2H) 7.33 (m, 2H) 7.18 (m, 3H) 6.56 (s, 1H) 5.51 (d, 1H) 5.14 (d, 1H) 3.54 (d, 2H) 3.10 (m, 2H) 2.85 (m, 1H) 2.83 (d, 3H) 2.06 (d, 2H) 1.85 (m, 2H); MS m/z: [M+1]⁺, 557.2.

### Compound 2 2-(3-Fluorophenyl)-2-(6-(4-(4-methylpiperazin-1-yl)phenyl)-4-oxo-2H-benzo[e][1,3]oxazin-3 (4H)-yl)-N-(thiazol-2-yl)acetamide

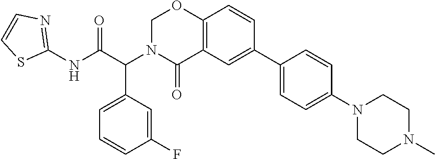 [SMILES: CN1CCN(c2ccc(-c3ccc4c(c3)C(=O)N(C(C(=O)Nc3nccs3)c3cccc(F)c3)CO4)cc2)CC1]

Compound 2 was prepared in a similar manner to compound 1, from 2-(3-fluorophenyl)-2-(6-(4-(1-methylpiperidin-4-yl)phenyl)-4-oxo-2H-benzo[e][1,3]oxazin-3 (4H)-yl)-N-(thiazol-2-yl)acetamide and 1-methyl-4-(4-(4,4,5,5-tetramethyl-1,3,2-dioxaborolan-2-yl)phenyl) piperazine. ¹H NMR (500 MHZ, DMSO-d₆) δ: 9.64 (br s, 1H) 8.03 (d, 1H) 7.84 (dd, 1H) 7.59 (d, 2H) 7.55 (m, 1H) 7.53 (d, 1H) 7.34 (d, 1H) 7.31 (m, 1H) 7.18 (d, 2H) 7.14 (d, 1H) 7.11 (d, 2H) 6.56 (s, 1H) 5.49 (d, 1H) 5.12 (d, 1H) 3.93 (d, 2H) 3.54 (d, 2H) 3.18 (m, 2H) 3.00 (m, 2H) 2.87 (d, 3H); MS m/z: [M+1]⁺, 558.2.

Compounds 3-5 were prepared in a similar manner to compound 1, from methyl 2-amino-2-(3-fluorophenyl)acetate and the corresponding benzoic acid.

| | | m/z | ¹H NMR | |
|---|---|---|---|---|
| Cmpd | Structure/Name | [M + 1]⁺ | (DMSO-d₆) δ | Starting acid |
| 3 | 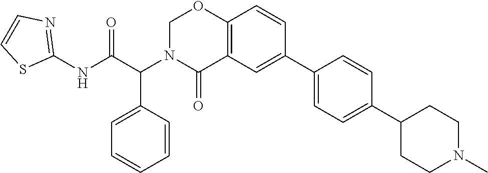 [SMILES: CN1CCC(c2ccc(-c3ccc4c(c3)C(=O)N(C(C(=O)Nc3nccs3)c3ccccc3)CO4)cc2)CC1] | 538.9 | 9.26 (br s, 1H) 8.08 (d, 1H) 7.86 (dd, 1H) 7.66 (d, 2H) 7.52 (d, 1H) 7.49 (m, 3H) 7.34 (m, 4H) 7.16 (d, 1H) 6.60 (s, 1H) 5.51 (d, 1H) 5.06 (d, 1H) | 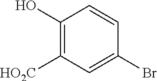 [SMILES: O=C(O)c1cc(Br)ccc1O] |
| | | | 3.54 (d, 2H) | |
| | 2-(6-(4-(1-Methylpiperidin-4- | | 3.10 (m, 2H) | |
| | yl)phenyl)-4-oxo-2H- | | 2.85 (m, 1H) | |
| | benzo[e][1,3]oxazin-3(4H)-yl)-2- | | 2.84 (d, 3H) | |
| | phenyl-N-(thiazol-2-yl)acetamide | | 2.07 (d, 2H) | |
| | | | 1.87 (m, 2H) | |
| 4 | 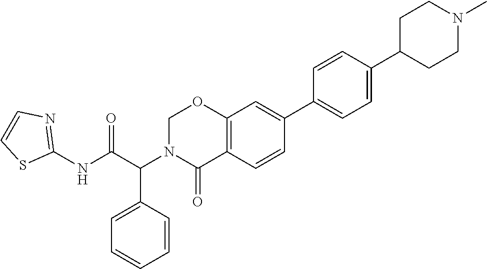 [SMILES: CN1CCC(c2ccc(-c3ccc4c(c3)OCN(C(C(=O)Nc3nccs3)c3ccccc3)C4=O)cc2)CC1] | 539.1 | 9.59 (br s, 1H) 7.94 (d, 1H) 7.72 (d, 2H) 7.53 (d, 1H) 7.50 (m, 4H) 7.36 (d, 2H) 7.33 (m, 4H) 6.59 (s, 1H) 5.51 (d, 1H) 5.06 (d, 1H) 3.54 (d, 2H) 3.10 (m, 2H) 2.86 (m, 1H) 2.84 (d, 3H) 2.05 (d, 2H) 1.87 (m, 2H) | 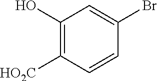 [SMILES: O=C(O)c1ccc(Br)cc1O] |
| | 2-(7-(4-(1-Methylpiperidin-4- | | | |
| | yl)phenyl)-4-oxo-2H- | | | |
| | benzo[e][1,3]oxazin-3(4H)-yl)-2- | | | |
| | phenyl-N-(thiazol-2-yl)acetamide | | | |
| 5 | 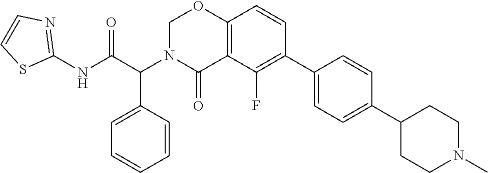 [SMILES: CN1CCC(c2ccc(-c3ccc4c(c3F)C(=O)N(C(C(=O)Nc3nccs3)c3ccccc3)CO4)cc2)CC1] | 557.4 | 9.33 (br s, 1H) 7.68 (m, 1H) 7.53 (d, 1H) 7.50 (m, 5H) 7.37 (d, 2H) 7.34 (m, 3H) 7.02 (d, 1H) 6.60 (s, 1H) 5.46 (d, 1H) 5.09 (d, 1H) 3.55 (d, | 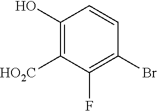 [SMILES: O=C(O)c1c(O)ccc(Br)c1F] |
| | | | 2H) 3.10 (m, | |
| | 2-(5-Fluoro-6-(4-(1- | | 2H) 2.86 (m, | |
| | methylpiperidin-4-yl)phenyl)-4- | | 1H) 2.83 (d, | |
| | oxo-2H-benzo[e][1,3]oxazin- | | 2H) 2.08 (d, | |
| | 3(4H)-yl)-2-phenyl-N-(thiazol-2- | | 2H) 1.86 (m, | |
| | yl)acetamide | | 2H) | |

### 3-Bromo-2-fluoro-6-hydroxybenzoic acid

Boron tribromide (1.74 mL, 18.1 mmol) was added dropwise to a solution of 3-bromo-2-fluoro-6-methoxybenzoic acid (1.5 g, 6.02 mmol) in dichloromethane (17 mL) at −78° C. under nitrogen and stirred for 1 hour, and then at room temperature for 1 hour. The reaction was quenched with water, stirred for 10 mins, then poured into brine (10 mL), and extracted with DCM (3×30 mL). Combined organic extracts were washed with brine, dried with MgSO₄, and concentrated under reduced pressure to give the title compound (742 mg, 53%). ¹H NMR (500 MHZ, DMSO-d₆) δ: 7.61 (m, 1H) 6.75 (d, 1H); MS m/z: [M+1]⁺, 236.9.

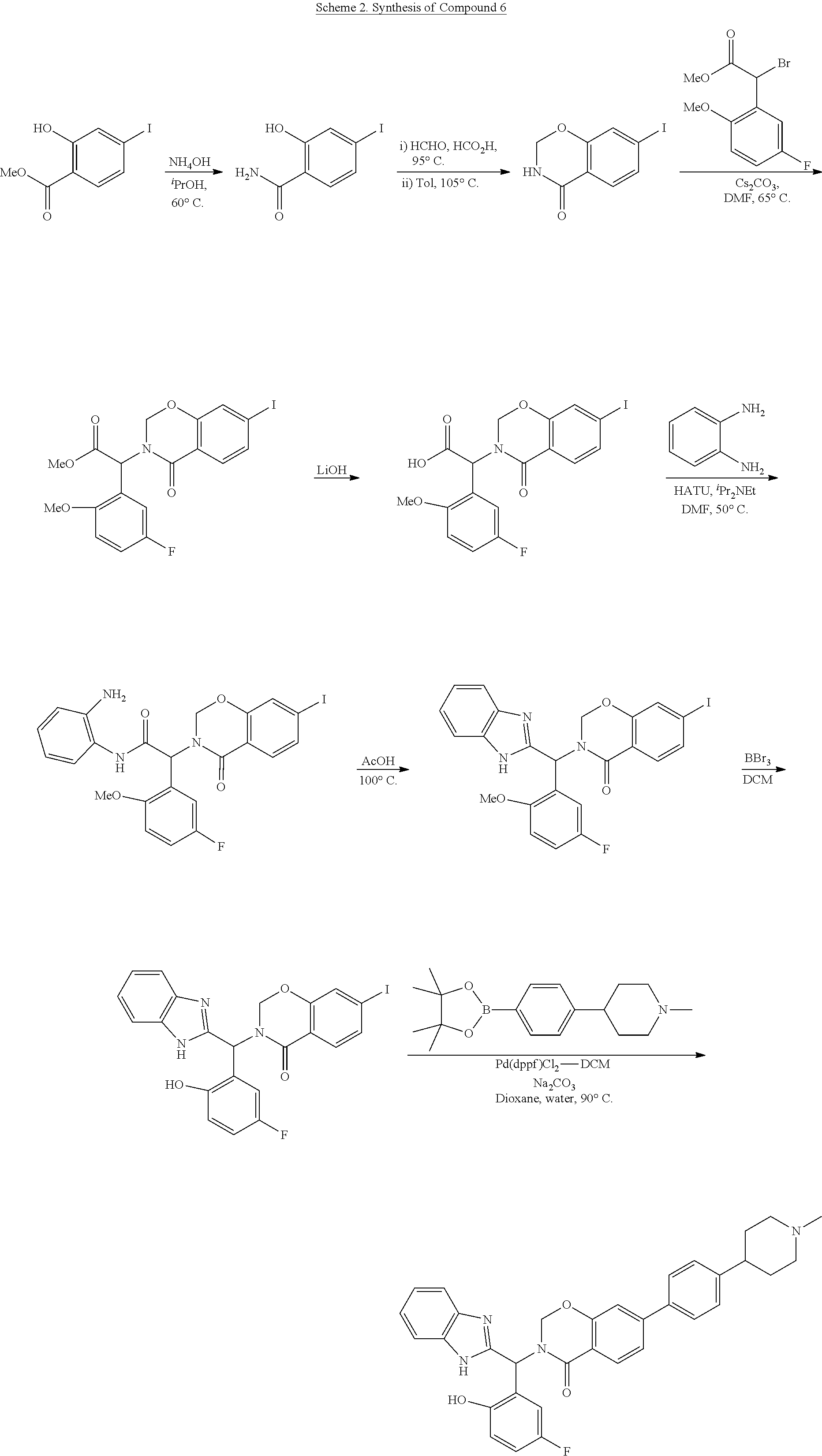

### 2-Hydroxy-4-iodobenzamide

A mixture of methyl 2-hydroxy-4-iodobenzamide (3.0 g, 10.79 mmol) and ammonium hydroxide (8.5 mL, 269.7 mmol) in isopropanol (10 mL) was stirred at 60° C. for 5 hours. After cooling, the reaction mixture was concentrated under reduced pressure, added to saturated brine (10 mL), and extracted with EtOAc (3×20 mL). Combined organic extracts were washed with saturated brine, dried over MgSO₄, filtered, concentrated under reduced pressure, and the residue was purified by silica chromatography (0-50% EtOAc/hexane) to give the title compound (1.97 g, 70%). MS m/z: [M+1]⁺, 263.7.

### 7-Iodo-2,3-dihydro-4H-benzo[e][1,3]oxazin-4-one

A mixture of 2-hydroxy-4-iodobenzamide (1.8 g, 6.84 mmol), formic acid (6 mL), and 37% formaldehyde (6 mL) was stirred at 95° C. for 2 hours. After cooling, the reaction mixture was neutralized with saturated aqueous Na₂CO₃, and extracted with EtOAc (3×20 mL). Combined organic extracts were washed with saturated brine, dried over MgSO₄, filtered, concentrated under reduced pressure, and the residue was purified by silica chromatography (0-85% EtOAc/hexane). The intermediate product was dissolved in toluene (25 mL) and stirred at 105° C. for 3.5 hours, then concentrated under reduced pressure to give the title compound (750 mg, 40%). ¹H NMR (500 MHZ, DMSO-d₆) δ: 10.19 (br s, 1H) 7.57 (d, 1H) 7.23 (s, 1H) 7.09 (d, 1H) 4.83 (d, 2H); MS m/z: [M+1]⁺, 276.0.

### Methyl 2-(5-fluoro-2-methoxyphenyl)-2-(7-iodo-4-oxo-2H-benzo[e][1,3]oxazin-3 (4H)-yl)acetate

A mixture of 7-iodo-2,3-dihydro-4H-benzo[e][1,3]oxazin-4-one (610 mg, 2.22 mmol), methyl 2-bromo-2-(5-fluoro-2-methoxyphenyl)acetate (645 mg, 2.33 mmol), and Cs₂CO₃ (1.45 g, 4.44 mmol) in DMF (15 mL) was stirred at 65° C. for 1 hour. Another aliquot of methyl 2-bromo-2-(5-fluoro-2-methoxyphenyl)acetate (100 mg, 0.36 mmol) was added and reaction mixture was heated for another 15 min. After cooling, the reaction mixture was poured into saturated brine (10 mL) and extracted with EtOAc (3×20 mL). The combined organic extracts were washed with saturated brine, dried over MgSO₄, filtered, concentrated under reduced pressure, and purified by silica chromatography (0-40% EtOAc/hexane) to give the title compound (248 mg, 24%). ¹H NMR (500 MHZ, DMSO-d₆) δ: 7.57 (m, 2H) 7.50 (d, 1H) 7.26 (m, 1H) 7.15 (m, 2H) 6.26 (s, 1H) 5.41 (d, 1H) 5.14 (d, 1H) 3.77 (s, 3H) 3.72 (s, 3H); MS m/z: [M+1]⁺, 472.1.

### 2-(5-Fluoro-2-methoxyphenyl)-2-(7-iodo-4-oxo-2H-benzo[e][1,3]oxazin-3 (4H)-yl)acetic acid

A mixture of methyl 2-(5-fluoro-2-methoxyphenyl)-2-(7-iodo-4-oxo-2H-benzo[e][1,3]oxazin-3 (4H)-yl)acetate (245 mg, 0.52 mmol) and lithium hydroxide monohydrate (33 mg, 0.78 mmol) in water (1 mL), methanol (1 mL), and THF (1 mL) was stirred at room temperature for 30 mins. The reaction mixture was concentrated under reduced pressure, then poured into water and brought to pH 2 with hydrochloric acid (1.5 N). The mixture was filtered to give the title compound (205 mg, 86%). ¹H NMR (500 MHZ, DMSO-d₆) δ: 7.57 (s, 1H) 7.56 (d, 1H) 7.49 (d, 1H) 7.25 (m, 1H) 7.13 (m, 1H) 7.12 (m, 1H) 6.19 (s, 1H) 5.41 (d, 1H) 5.15 (d, 1H) 3.76 (s, 3H); MS m/z: [M+1]⁺, 457.7.

### N-(2-Aminophenyl)-2-(5-fluoro-2-methoxyphenyl)-2-(7-iodo-4-oxo-2H-benzo[e][1,3]oxazin-3 (4H)-yl)acetamide

Diisopropylethylamine (0.234 mL, 1.35 mmol) was added to a mixture of 2-(5-fluoro-2-methoxyphenyl)-2-(7-iodo-4-oxo-2H-benzo[e][1,3]oxazin-3 (4H)-yl)acetic acid (205 mg, 0.448 mmol), 1,2-phenylenediame (97 mg, 0.897 mmol) and HATU (341 mg, 0.897 mmol) in DMF (3 mL), and stirred at 50° C. for 3 hours. After cooling, the reaction mixture was poured into saturated brine (10 mL) and extracted with EtOAc (3×20 mL). The combined organic extracts were washed with saturated brine, dried over MgSO₄, filtered, concentrated under reduced pressure, and purified by reverse phase HPLC eluting with 0-80% ACN/H₂O (0.038% TFA modifier) to give the title compound, carried into the next step. ¹H NMR (500 MHz, DMSO-d₆) δ: 9.73 (s, 1H) 7.61 (d, 1H) 7.57 (dd, 1H) 7.48 (d, 1H) 7.28 (m, 1H) 7.19 (dd, 1H) 7.16 (dd, 1H) 7.13 (dd, 1H) 6.95 (m, 1H) 6.75 (dd, 1H) 6.59 (m, 1H) 6.51 (s, 1H) 5.42 (d, 1H) 5.09 (d, 1H) 3.74 (s, 3H); MS m/z: [M+1]⁺, 547.7.

### 3-((1H-Benzo[d]imidazol-2-yl) (5-fluoro-2-methoxyphenyl)methyl)-7-iodo-2,3-dihydro-4H-benzo[e][1,3]oxazin-4-one

A solution of N-(2-aminophenyl)-2-(5-fluoro-2-methoxyphenyl)-2-(7-iodo-4-oxo-2H-benzo[e][1,3]oxazin-3 (4H)-yl)acetamide in acetic acid (10 mL) was heated at 100° C. for 1 hour. The reaction mixture was concentrated under reduced pressure to give the title compound (98 mg, 40% over 2 steps). ¹H NMR (500 MHZ, DMSO-d₆) δ: 7.60 (m, 4H) 7.53 (s, 1H) 7.28 (m, 3H) 7.19 (s, 1H) 7.17 (dd, 1H) 6.94 (d, 1H) 5.52 (d, 1H) 5.34 (d, 1H) 3.72 (s, 3H); MS m/z: [M+1]⁺, 529.7.

### 3-((1H-Benzo[d]imidazol-2-yl) (5-fluoro-2-hydroxyphenyl)methyl)-7-iodo-2,3-dihydro-4H-benzo[e][1,3]oxazin-4-one

Boron tribromide (0.034 mL, 0.357 mmol) was added dropwise to a solution of 3-((1H-benzo[d]imidazol-2-yl) (5-fluoro-2-methoxyphenyl)methyl)-7-iodo-2,3-dihydro-4H-benzo[e][1,3]oxazin-4-one (98 mg, 0.178 mmol) in dichloromethane (2 mL) at −78° C. and stirred for 2 hours. The reaction was then stirred at RT overnight. Another aliquot of boron tribromide (0.069 mL, 0.714 mmol) was added and the mixture stirred for 7 hours at RT. The reaction was quenched with water, poured into saturated brine (10 mL), and extracted with DCM (3×30 mL). Combined organic extracts were washed with saturated brine, dried with MgSO₄, and concentrated under reduced pressure to give the title compound (45 mg, 49%). MS (m/z): [M+1]⁺, 516.2.

### 3-((1H-Benzo[d]imidazol-2-yl) (5-fluoro-2-hydroxyphenyl)methyl)-7-(4-(1-methylpiperidin-4-yl)phenyl)-2,3-dihydro-4H-benzo[e][1,3]oxazin-4-one

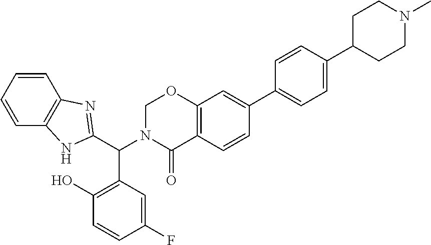 [SMILES: CN1CCC(c2ccc(-c3ccc4c(c3)OCN(C(c3nc5ccccc5[nH]3)c3cc(F)ccc3O)C4=O)cc2)CC1]

A mixture of 3-((1H-benzo[d]imidazol-2-yl) (5-fluoro-2-hydroxyphenyl)methyl)-7-iodo-2,3-dihydro-4H-benzo[e][1,3]oxazin-4-one (45 mg, 0.0873 mmol), 1-methyl-4-(4-(4,4,5,5-tetramethyl-1,3,2-dioxaborolan-2-yl)phenyl) piperidine (289 mg, 0.96 mmol), Pd(dppf)Cl₂-DCM (14 mg, 0.0175 mmol), and Na₂CO₃ (28 mg, 0.262 mmol) in dioxane (3.75 mL) and water (1.2 mL) was heated under N₂ in a sealed vial at 90° C. for 17.5 hours. After cooling, the reaction mixture was filtered through a syringe filter, and purified by reverse phase HPLC eluting with 0-80% ACN/H₂O (0.038% TFA modifier) to give Compound 006 (6.5 mg, 13%). ¹H NMR (500 MHZ, DMSO-d₆) δ: 9.98 (s, 1H) 9.28 (br s, 1H) 7.91 (d, 1H) 7.73 (d, 2H) 7.58 (m, 2H) 7.49 (dd, 1H) 7.37 (d, 2H) 7.34 (d, 1H) 7.24 (m, 2H) 7.20 (s, 1H) 7.10 (m, 1H) 6.91 (dd, 1H), 6.79 (d, 1H) 5.58 (d, 1H) 5.38 (d, 1H) 3.54 (d, 2H) 3.10 (m, 2H) 2.86 (m, 1H) 2.84 (d, 3H) 2.06 (d, 2H) 1.84 (m, 2H); MS m/z: [M+1]⁺, 563.0. Methyl 2-bromo-2-(5-fluoro-2-methoxyphenyl)acetate was prepared according to the procedures described in WO 2021/096948.

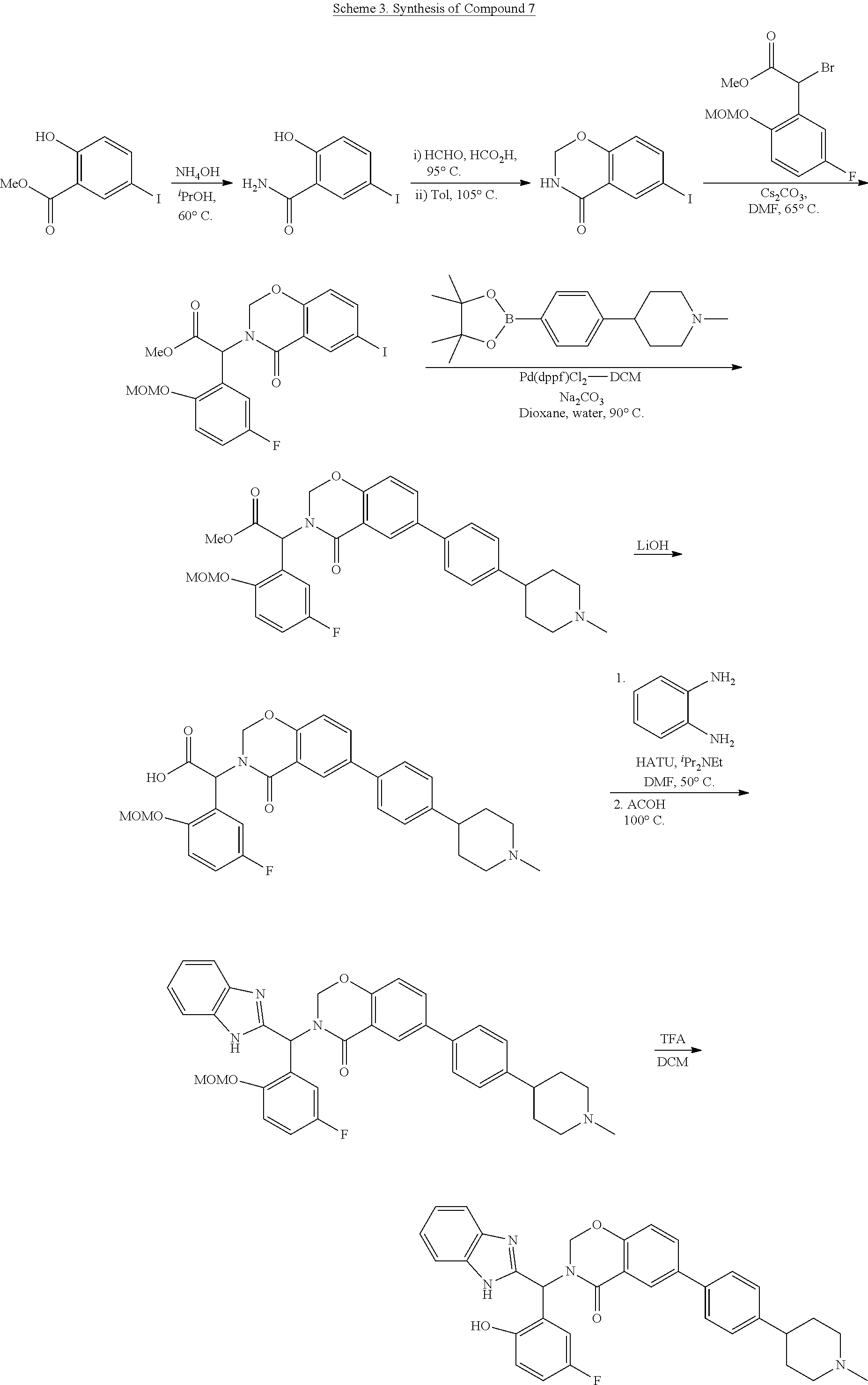

### 2-Hydroxy-5-iodobenzamide

A mixture of methyl 2-hydroxy-5-iodobenzamide (3.0 g, 10.79 mmol) and ammonium hydroxide (8.5 mL, 269.7 mmol) in isopropanol (10 mL) was stirred at 60° C. for 2 hours. After cooling, the reaction mixture was degassed with nitrogen, water was added and the resulting precipitate was filtered and dried in a vacuum oven to give the title compound as a white solid (2.82 g, 99.6%). MS (m/z): [M+1]⁺, 263.74.

### 6-Iodo-2,3-dihydro-4H-benzo[e][1,3]oxazin-4-one

A mixture of 2-hydroxy-5-iodobenzamide (2.82 g, 10.7 mmol), formic acid (15 mL), and 37% formaldehyde (15 mL) was stirred at 95° C. for 1 hour. After cooling, the reaction mixture was quenched with water and extracted with EtOAc (3×40 mL). Combined organic extracts were washed with saturated brine, dried over MgSO₄, filtered, concentrated under reduced pressure, and the residue was purified by normal phase flash chromatography (0-60% EtOAc/hexane) to give the title compound (368 mg) and 3-(hydroxymethyl)-7-iodo-2,3-dihydro-4H-benzo[e][1,3]oxazin-4-one as a byproduct. This byproduct was dissolved in toluene (25 mL) and stirred at 105° C. overnight, then concentrated under reduced pressure to give an additional batch of the title compound as a white solid, combined with the first batch (818 mg, 28%). ¹H NMR (500 MHz, DMSO-d₆) δ: 8.74 (br s, 1H) 8.00 (d, 1H) 7.82 (dd, 1H) 6.91 (d, 1H) 5.17 (d, 2H); MS (m/z): [M+1]⁺, 275.88.

### Methyl 2-(5-fluoro-2-(methoxymethoxy)phenyl)-2-(6-iodo-4-oxo-2H-benzo[e][1,3]oxazin-3 (4H)-yl)acetate

A mixture of 6-iodo-2,3-dihydro-4H-benzo[e][1,3]oxazin-4-one (0.818 g, 2.76 mmol), methyl 2-bromo-2-(5-fluoro-2-(methoxymethoxy)phenyl)acetate (1.06 g, 3.32 mmol), and cesium carbonate (1.80 g, 5.52 mmol) in DMF (20 mL) was stirred at room temperature for 2 hours. The reaction mixture was poured into saturated brine (30 mL) and extracted with EtOAc (3×40 mL). The combined organic extracts were washed with saturated brine, dried over MgSO₄, filtered, concentrated under reduced pressure, and purified by normal phase flash chromatography (0-70% EtOAc/hexane) to give the title compound as a colorless oil (362 mg, 26%). ¹H NMR (500 MHZ, DMSO-d₆) δ: 8.09 (d, 1H) 7.86 (dd, 1H) 7.25 (m, 1H) 7.19 (m, 2H) 6.91 (d, 1H) 6.32 (s, 1H) 5.43 (d, 1H) 5.21 (d, 1H) 5.20 (d, 1H) 5.16 (d, 1H) 3.74 (s, 3H) 3.31 (s, 3H); MS (m/z): [M+1]⁺, 501.89.

### Methyl 2-(5-fluoro-2-(methoxymethoxy)phenyl)-2-(6-(4-(1-methylpiperidin-4-yl)phenyl)-4-oxo-2H-benzo[e][1,3]oxazin-3 (4H)-yl)acetate

A mixture of methyl 2-(5-fluoro-2-(methoxymethoxy)phenyl)-2-(6-iodo-4-oxo-2H-benzo[e][1,3]oxazin-3 (4H)-yl)acetate (0.362 g, 0.722 mmol), 1-methyl-4-(4-(4,4,5,5-tetramethyl-1,3,2-dioxaborolan-2-yl)phenyl) piperidine (0.433 g, 1.44 mmol), Pd(dppf)Cl₂-DCM (0.118 g, 0.144 mmol), and Na₂CO₃ (0.229 g, 2.16 mmol) in dioxane (5 mL) and water (1.6 mL) was heated under N₂ in a sealed vial at 90° C. for 30 minutes. After cooling, the reaction mixture was poured into saturated brine (10 mL) and extracted with EtOAc (3×20 mL). The combined organic extracted were washed with saturated brine, dried over MgSO₄, filtered, concentrated under reduced pressure, and purified by reverse phase HPLC eluting with 0-80% ACN/H₂O (0.038% TFA modifier) to give the title compound as a white solid (240 mg, 61%). ¹H NMR (500 MHZ, DMSO-d₆) δ: 9.27 (br s, 1H) 8.06 (d, 1H) 7.86 (dd, 1H) 7.66 (d, 2H) 7.35 (d, 2H) 7.26 (m, 1H) 7.20 (m, 2H) 7.16 (d, 1H) 6.36 (s, 1H) 5.47 (d, 1H) 5.23 (d, 1H) 5.22 (d, 1H) 5.18 (d, 1H) 3.75 (s, 3H) 3.54 (d, 2H) 3.10 (m, 2H) 2.85 (m, 1H) 2.84 (d, 3H) 2.06 (d, 2H) 1.85 (m, 2H); MS (m/z): [M+1]⁺, 549.19.

### 2-(5-Fluoro-2-(methoxymethoxy)phenyl)-2-(6-(4-(1-methylpiperidin-4-yl)phenyl)-4-oxo-2H-benzo[e][1,3]oxazin-3 (4H)-yl)acetic acid

A mixture of methyl 2-(5-fluoro-2-(methoxymethoxy)phenyl)-2-(6-(4-(1-methylpiperidin-4-yl)phenyl)-4-oxo-2H-benzo[e][1,3]oxazin-3 (4H)-yl)acetate (0.240 g, 0.437 mmol) and lithium hydroxide monohydrate (0.028 g, 0.656 mmol) in water (2 mL), methanol (2 mL), and THF (2 mL) was stirred at room temperature for 30 minutes. Another aliquot of lithium hydroxide (0.028 g, 0.656 mmol) was added and the reaction stirred for 30 minutes. The reaction mixture was concentrated under reduced pressure, then poured into water and brought to pH 7 with hydrochloric acid (1 N). The mixture was filtered to recover the title compound as a purple solid (100 mg, 43%). MS (m/z): [M+1]⁺, 535.16.

### 3-((1H-Benzo[d]imidazol-2-yl) (5-fluoro-2-(methoxymethoxy)phenyl)methyl)-6-(4-(1-methylpiperidin-4-yl)phenyl)-2,3-dihydro-4H-benzo[e][1,3]oxazin-4-one

DIEA (0.163 mL, 0.935 mmol) was added to a mixture of 2-(5-fluoro-2-(methoxymethoxy)phenyl)-2-(6-(4-(1-methylpiperidin-4-yl)phenyl)-4-oxo-2H-benzo[e][1,3]oxazin-3 (4H)-yl)acetic acid (0.100 g, 0.187 mmol), 1,2-phenylenediamine (0.040 g, 0.374 mmol) and HATU (0.142 g, 0.374 mmol) in DMF (3 mL), and stirred at room temperature for 30 minutes. The reaction mixture was poured into saturated brine (10 mL) and extracted with EtOAc (3×10 mL). The combined organic extracts were washed with saturated brine, dried over MgSO₄, filtered, concentrated under reduced pressure. The crude material was carried directly to the next step without further purification. MS (m/z): [M+1]⁺, 625.53.

A solution of the above material in acetic acid (6 mL) was heated at 65° C. for 1 hour. The reaction mixture was concentrated under reduced pressure and carried directly to the next step without further purification. MS (m/z): [M+1]⁺, 607.26.

### 3-((1H-Benzo[d]imidazol-2-yl) (5-fluoro-2-hydroxyphenyl)methyl)-6-(4-(1-methylpiperidin-4-yl)phenyl)-2,3-dihydro-4H-benzo[e][1,3]oxazin-4-one 2,2,2-trifluoroacetate

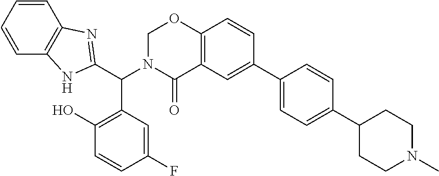 [SMILES: CN1CCC(c2ccc(-c3ccc4c(c3)C(=O)N(C(c3nc5ccccc5[nH]3)c3cc(F)ccc3O)CO4)cc2)CC1]

A solution of 3-((1H-benzo[d]imidazol-2-yl) (5-fluoro-2-(methoxymethoxy)phenyl)methyl)-6-(4-(1-methylpiperidin-4-yl)phenyl)-2,3-dihydro-4H-benzo[e][1,3]oxazin-4-one in DCM (2 mL) and TFA (2 mL) was stirred at room temperature overnight. The reaction mixture was concentrated under reduced pressure and purified by reverse phase HPLC eluting with 0-80% ACN/H₂O (0.038% TFA modifier) to give Compound 007, as the TFA salt (31 mg, 24% yield from the acid) as a white solid. ¹H NMR (500 MHZ, DMSO-d₆) δ: 10.02 (br s, 1H) 9.30 (br s, 1H) 8.06 (d, 1H) 7.86 (dd, 1H) 7.67 (d, 2H) 7.59 (m, 2H) 7.35 (d, 2H) 7.26 (m, 2H) 7.20 (s, 1H) 7.17 (d, 1H) 7.13 (m, 1H) 6.92 (dd, 1H) 6.81 (m, 1H) 5.59 (d, 1H) 5.37 (d, 1H) 3.54 (d, 2H) 3.12 (m, 2H) 2.84 (m, 4H) 2.06 (d, 2H) 1.84 (m, 2H); MS (m/z): [M+1]⁺, 563.19. Methyl 2-bromo-2-(5-fluoro-2-(methoxymethoxy)phenyl)acetate was prepared according to the procedures described in WO 2021096948.

### Example 2: HTRF-Based EGFR Biochemical Assays

EGFR biochemical activity measurements were carried out using the homogeneous time-resolved fluorescence (HTRF) assay (Cisbio). Inhibitors and DMSO normalizations were first dispensed to empty black low-volume 384-well plates (Corning) with D300 digital liquid dispenser (HP). All reactions were carried out at room temperature and solutions were added to plates with a Multidrop Combi Reagent Dispenser (ThermoFisher). The reaction mixture (10 UL final volume) contained 1 μM tyrosine kinase peptide-biotin substrate and mutant EGFR in a reaction buffer (50 mM HEPES pH 7.0, 5 mM MgCl₂, 1 mM MnCl₂, 0.01% BSA, 2 mM TCEP, 0.1 mM NaVO₄). Enzyme concentrations were adjusted to accommodate varying kinase activities (L858R 0.1 nM, L858R/T790M 0.02 nM). Enzyme reaction solution (2× concentrations, 5 μL) was added to 384-well plates containing compounds and incubated for 30 mins. Enzyme reactions were initiated with the addition of 5 μL of ATP to a final concentration of 100 μM and reacted for 20 mins. Reactions were quenched with the addition of 10 μL of phospho-tyrosine antibody-Europium (III) cryptate (1-to-180 volume ratio) and Streptavidin-XL665 (46.7 nM) in EDTA-containing detection buffer, then incubated at room temperature for 1 hour, and read with a PHERAstar plate reader (excitation=337 nm, emission=620 nm and 665 nm). IC₅₀ values were determined by inhibition curves (11-point curves from 1.0 μM to 0.130 nM or 23-point curves from 1.0 μM to 0.130 μM) in triplicate with non-linear least squares fit in GraphPad Prism 7.0d. The data obtained are shown in Table 3 below.

TABLE 3

| No. | LRTM enzyme IC50 (nM) | LR enzyme IC50 (nM) |
|---|---|---|
| 001 | 0.4 | 2.8 |
| 002 | 4 | 23 |
| 003 | 0.8 | 23 |
| 004 | 21 | >100 |
| 005 | 1 | 3.7 |
| 006 | 0.9 | 3.6 |
| 007 | 0.7 | 1.9 |

### Example 3: Ba/F3 Cell Proliferation Models

The EGFR mutant L858R and L858R/T790M Ba/F3 cells have been previously described (Zhou, W., et al. Nature 462, 2009, 1070-1074). All cell lines were maintained in RPMI 1640 (Cellgro; Mediatech Inc., Herndon, CA) supplemented with 10% FBS, 100 units/mL penicillin, 100 units/mL streptomycin. The EGFR 1941R mutation was introduced via site directed mutagenesis using the Quick Change Site-Directed Mutagenesis kit (Stratagene; La Jolla, CA) according to the manufacturer's instructions. All constructs were confirmed by DNA sequencing. The constructs were shuttled into the retroviral vector JP1540 using the Cre-recombination system (Agilent Technologies, Santa Clara, CA). Ba/F3 cells were then infected with retrovirus per standard protocols, as described previously (Zhou, et al, Nature 2009). Stable clones were obtained by selection in puromycin (2 μg/ml).

Growth and inhibition of growth was assessed by the Cell Titer Glo assay (Promega, Madison, WI) and was performed according to the manufacturer's instructions. The Cell Titer Glo assay is a luminescence-based method used to determine the number of viable cells based on quantitation of the ATP present, which is directly proportional to the amount of metabolically active cells present. Ba/F3 cells of different EGFR genotypes were exposed to the compounds disclosed herein for 72 hours and the number of cells used per experiment was determined empirically as has been previously established (Zhou, et al., Nature 2009). All experimental points were set up in triplicates in 384-well plates and all experiments were repeated at least three times. The luminescent signal was detected using a spectrometer and the data was graphically displayed using GraphPad Prism version 5.0 for Windows, (GraphPad Software; www.graphpad.com). The curves were fitted using a non-linear regression model with a sigmoidal dose response. The results of this assay for the compounds disclosed herein are shown in Table 4 below.

TABLE 4

| No. | LRTM BaF3 cell IC₅₀ (uM) | LR BaF3 cell IC₅₀ (uM) |
|---|---|---|
| 001 | 0.51 | 1.48 |
| 003 | 0.46 | 1.25 |
| 005 | 0.46 | 1.04 |
| 006 | 0.88 | 1.54 |
| 007 | 0.22 | 0.47 |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1.-39. (canceled)

40. A compound of Formula I: 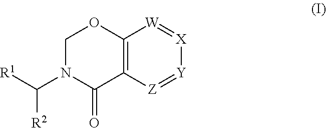
or a pharmaceutically acceptable salt thereof;
wherein:
W and Z are each, independently, N, CH, C-halo, C—(C₁-C₃ alkyl), or C—(C₁-C₃ alkoxy);
X and Y are each, independently, N, CH, or CR³;
provided that at least one of W, X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl, all of which are optionally substituted with one, two, or three R⁸;
R² is selected from the group consisting of 6-10 membered aryl, 5-10 membered heteroaryl, 3-10 membered heterocycloalkyl, and 3-10 membered cycloalkyl, all of which are optionally substituted with one, two, or three R⁶;
R³ is independently, at each occurrence, selected from the group consisting of halogen, OR⁴, NR⁴R⁴, SO₂R⁴, SO₂NHR⁴, NHSO₂R⁴, C(O)OR⁴, C(O)NHR⁴, C(O)R⁴, C₁-C₆ alkyl, C₂-C₆ alkenyl, C₂-C₆ alkynyl, 3-7 membered cycloalkyl, C₄-C₇ cycloalkenyl, C₆-C₁₀ aryl, 5-6 membered heteroaryl, and 5-7 membered heterocyclyl, wherein alkyl, alkenyl, or alkynyl are each optionally substituted one, two, or three times with R⁴, and wherein aryl, heteroaryl, or heterocyclyl are each optionally substituted one, two, or three times with R⁵;
R⁴ is independently, at each occurrence, selected from the group consisting of H, (CH₂)₀₋₃—(C₃-C₇ cycloalkyl), (CH₂)₀₋₃—(C₄-C₇ cycloalkenyl), (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁵;
R⁵ is independently, at each occurrence, selected from the group consisting of C₁-C₆ alkyl, C₁-C₆ haloalkyl, C₁-C₆ alkoxy, C₁-C₆ haloalkoxy, C₁-C₃ alkylamine, 3-10 membered cycloalkyl, halogen, COOH, C(O)O(C₁-C₆ alkyl), O(CH₂)₁₋₃—OH, NH₂, NH(C₁-C₆ alkyl), N(C₁-C₆ alkyl)₂, OH, CN, (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN;
alternatively, two R⁶, together with the atoms to which they are attached, can form 5-10 membered heteroaryl, 6-10 membered aryl, 3-10 membered heterocycloalkyl, or 3-10 membered cycloalkyl;
R⁷ is independently, at each occurrence, selected from the group consisting of C₁-C₆ alkyl, C₁-C₆ haloalkyl, C₁-C₆ alkoxy, C₁-C₆ haloalkoxy, halogen, NH₂, NH(C₁-C₆ alkyl), N(C₁-C₆ alkyl)₂, SO₂NH₂, SO₂NH(C₁-C₆ alkyl), SO₂N(C₁-C₆ alkyl)₂, (CH₂)₁₋₂—OH, C(O)(CH₂)₁₋₂—OH, C(O)(C₁-C₆ alkyl), and C(O)O(C₁-C₆ alkyl);
alternatively, two R⁷, together with the atoms to which they are attached, can form 5-10 membered heteroaryl, 6-10 membered aryl, 3-10 membered heterocycloalkyl, or 3-10 membered cycloalkyl;
R⁸ is independently, at each occurrence, selected from the group consisting of hydrogen, C₁-C₃ alkyl, C₁-C₃ haloalkyl, C₁-C₃ alkoxy, C₁-C₃ haloalkoxy, C₁-C₃ alkylamine, 3-6 membered cycloalkyl, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN; and
R⁹ is selected from the group consisting of 6-10 membered aryl, 5-10 membered heteroaryl, 3-10 membered heterocycloalkyl, and 3-10 membered cycloalkyl.

41. The compound of claim 40, wherein
W and Z are each, independently, N, C-halo, or CH;
X and Y are each, independently, CH or CR³;
provided that at least one of W, X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl;
R² is selected from the group consisting of 6-10 membered aryl and 5-10 membered heteroaryl, both of which are optionally substituted with one, two, or three R⁶;
R³ is independently, at each occurrence, selected from the group consisting of C₆-C₁₀ aryl, 5-6 membered heteroaryl, and 5-7 membered heterocyclyl, wherein aryl, heteroaryl, or heterocyclyl are each optionally substituted one, two, or three times with R⁵;
R⁵ is independently, at each occurrence, selected from the group consisting of 3-10 membered cycloalkyl, (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, and OH;
R⁷ is independently, at each occurrence, selected from the group consisting of C₁-C₆ alkyl, C₁-C₆ haloalkyl, C₁-C₆ alkoxy, and halogen;
R⁸ is selected from the group consisting of hydrogen, C₁-C₃ alkyl, C₁-C₃ haloalkyl, and C₁-C₃ alkoxy; and
R⁹ is 5-10 membered heteroaryl.

42. The compound of claim 40, wherein the compound of Formula I is a compound of Formula IIa or IIb: 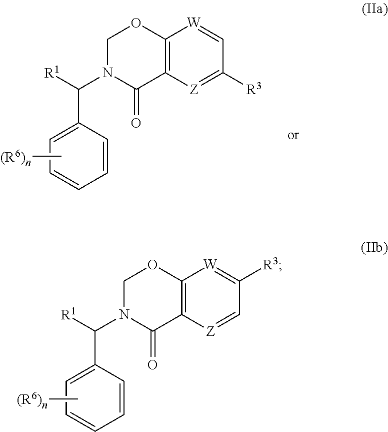
or a pharmaceutically acceptable salt thereof;
wherein n is 0, 1, or 2.

43. The compound of claim 42, wherein W is CH, Z is CH or C-halo, and R¹ is C(O)NHR⁹ or 5-10 membered heteroaryl.

44. The compound of claim 43, wherein R¹ is

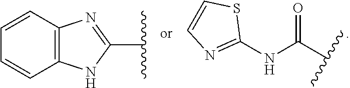

45. The compound of claim 44, wherein R³ is selected from the group consisting of

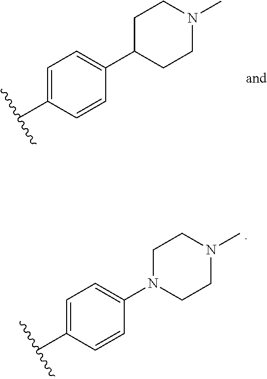 [SMILES: *c1ccc(C2CCN(C)CC2)cc1 ; *c1ccc(N2CCN(C)CC2)cc1]

46. The compound of claim 40, wherein the compound of Formula I is selected from the group consisting of: 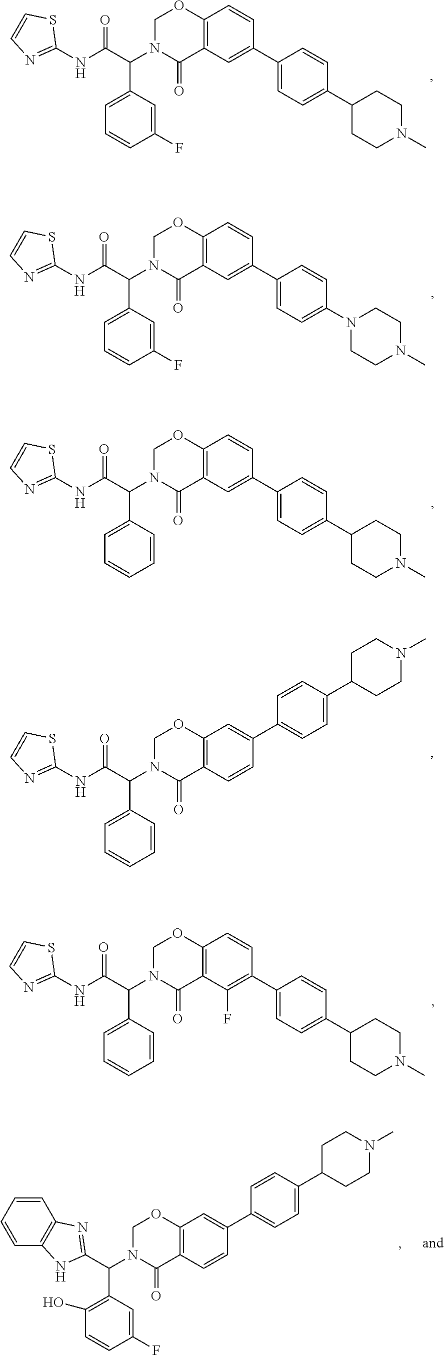 [SMILES: CN1CCC(c2ccc(-c3ccc4c(c3)C(=O)N(C(C(=O)Nc3nccs3)c3cccc(F)c3)CO4)cc2)CC1 ; CN1CCN(c2ccc(-c3ccc4c(c3)C(=O)N(C(C(=O)Nc3nccs3)c3cccc(F)c3)CO4)cc2)CC1 ; CN1CCC(c2ccc(-c3ccc4c(c3)C(=O)N(C(C(=O)Nc3nccs3)c3ccccc3)CO4)cc2)CC1 ; CN1CCC(c2ccc(-c3ccc4c(c3)OCN(C(C(=O)Nc3nccs3)c3ccccc3)C4=O)cc2)CC1 ; CN1CCC(c2ccc(-c3ccc4c(c3F)C(=O)N(C(C(=O)Nc3nccs3)c3ccccc3)CO4)cc2)CC1 ; CN1CCC(c2ccc(-c3ccc4c(c3)OCN(C(c3nc5ccccc5[nH]3)c3cc(F)ccc3O)C4=O)cc2)CC1] 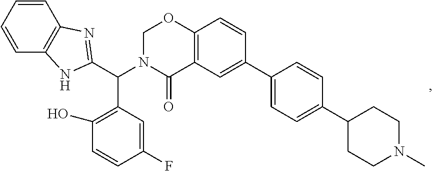 [SMILES: CN1CCC(c2ccc(-c3ccc4c(c3)C(=O)N(C(c3nc5ccccc5[nH]3)c3cc(F)ccc3O)CO4)cc2)CC1]
or a pharmaceutically acceptable salt thereof.

47. A pharmaceutical composition comprising a compound of claim 40, or a pharmaceutically acceptable salt thereof, and at least one pharmaceutically acceptable carrier.

48. A compound of Formula III: 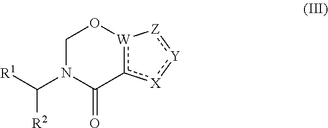
or a pharmaceutically acceptable salt thereof;
wherein [symbol]
represents an optional double bond;
W is N, C, or CH;
Z is selected from the group consisting of S, O, N, NH, CH₂, C-halo, and CH;
X and Y are each, independently, S, O, N, CH, NR³, or CR³;
provided that at least one of X, Y, or Z is CH;
R¹ is selected from the group consisting of C(O)NHR⁹, 6-10 membered aryl, 5-10 membered heteroaryl, 3-10 membered heterocycloalkyl, and 3-10 membered cycloalkyl, all of which are optionally substituted with one, two, or three R⁸;
R² is selected from the group consisting of 6-10 membered aryl, 5-10 membered heteroaryl, 3-10 membered heterocycloalkyl, and 3-10 membered cycloalkyl, all of which are optionally substituted with one, two, or three R⁶;
R³ is independently, at each occurrence, selected from the group consisting of halogen, OR⁴, NR⁴R⁴, SO₂R⁴, SO₂NHR⁴, NHSO₂R⁴, C(O)OR⁴, C(O)NHR⁴, C(O)R⁴, C₁-C₆ alkyl, C₂-C₆ alkenyl, C₂-C₆ alkynyl, 3-7 membered cycloalkyl, C₄-C₇ cycloalkenyl, C₆-C₁₀ aryl, 5-6 membered heteroaryl, and 5-7 membered heterocyclyl, wherein alkyl, alkenyl, or alkynyl are each optionally substituted one, two, or three times with R⁴, and wherein aryl, heteroaryl, or heterocyclyl are each optionally substituted one, two, or three times with R⁵;
R⁴ is independently, at each occurrence, selected from the group consisting of H, (CH₂)₀₋₃—(C₃-C₇ cycloalkyl), (CH₂)₀₋₃—(C₄-C₇ cycloalkenyl), (CH₂)₀₋₃—(C₆-C₁₀ aryl), (CH₂)₀₋₃-(5-6 membered heteroaryl), and (CH₂)₀₋₃-(5-7 membered heterocyclyl), wherein the aryl, heteroaryl, or heterocyclyl are each optionally substituted one, two, or three times with R⁵;
R⁵ is independently, at each occurrence, selected from the group consisting of C₁-C₆ alkyl, C₁-C₆ haloalkyl, C₁-C₆ alkoxy, C₁-C₆ haloalkoxy, C₁-C₃ alkylamine, 3-10 membered cycloalkyl, halogen, COOH, C(O)O(C₁-C₆ alkyl), O(CH₂)₁₋₃—OH, NH₂, NH(C₁-C₆ alkyl), N(C₁-C₆ alkyl)₂, OH, CN, (CH₂)₀₋₃—(C₆-C₁₀ aryl), (CH₂)₀₋₃-(5-6 membered heteroaryl), O(CH₂)₀₋₃-(4-7 membered heterocyclyl), and (CH₂)₀₋₃-(4-7 membered heterocyclyl), wherein the alkyl, alkoxy, aryl, heteroaryl, or heterocyclyl are each optionally substituted one, two, or three times with R⁷;
R⁶ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN;
alternatively, two R⁶, together with the atoms to which they are attached, can form 5-10 membered heteroaryl, 6-10 membered aryl, 3-10 membered heterocycloalkyl, or 3-10 membered cycloalkyl;
R⁷ is independently, at each occurrence, selected from the group consisting of substituents independently selected from C₁-C₆ alkyl, C₁-C₆ haloalkyl, C₁-C₆ alkoxy, C₁-C₆ haloalkoxy, halogen, NH₂, NH(C₁-C₆ alkyl), N(C₁-C₆ alkyl)₂, SO₂NH₂, SO₂NH(C₁-C₆ alkyl), SO₂N(C₁-C₆ alkyl)₂, (CH₂)₁₋₂—OH, C(O)(CH₂)₁₋₂—OH, C(O)(C₁-C₆ alkyl), and C(O)O(C₁-C₆ alkyl);
alternatively, two R⁷, together with the atoms to which they are attached, can form 5-10 membered heteroaryl, 6-10 membered aryl, 3-10 membered heterocycloalkyl, or 3-10 membered cycloalkyl;
R⁸ is independently, at each occurrence, selected from the group consisting of C₁-C₃ alkyl, C₁-C₃ haloalkyl, C₁-C₃ alkoxy, C₁-C₃ haloalkoxy, C₁-C₃ alkylamine, 3-6 membered cycloalkyl, halogen, OH, NO₂, NH₂, NH(C₁-C₆ alkyl), N(C₁-C₆ alkyl)₂, (CH₂)₁₋₄OH, S(O)₀₋₂H, S(O)₀₋₂NH₂, or CN; and
R⁹ is selected from the group consisting of 6-10 membered aryl, 5-10 membered heteroaryl, 3-10 membered heterocycloalkyl, and 3-10 membered cycloalkyl.

49. The compound of claim 48, wherein the compound of Formula III is a compound of Formula: 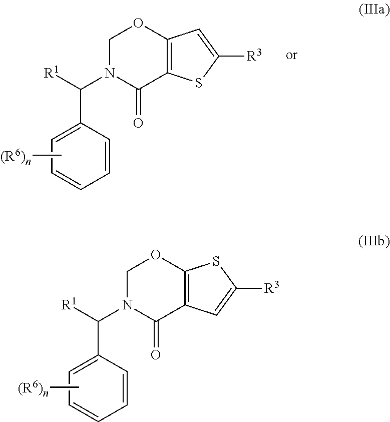
or a pharmaceutically acceptable salt thereof;
wherein n is 0, 1, or 2.

50. The compound of claim 49, wherein R¹ is

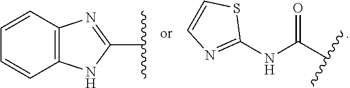

51. The compound of claim 50, wherein R³ is selected from the group consisting of

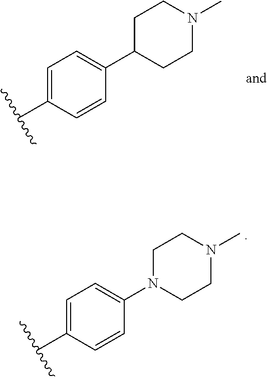 [SMILES: *c1ccc(C2CCN(C)CC2)cc1 ; *c1ccc(N2CCN(C)CC2)cc1]

52. The compound of claim 48, wherein the compound of Formula III is selected from the group consisting of: 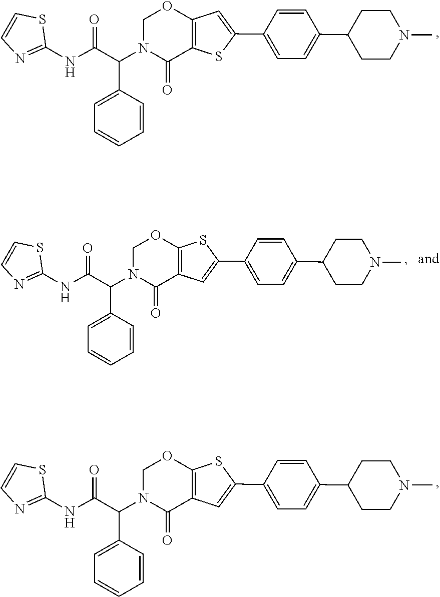 [SMILES: CN1CCC(c2ccc(-c3cc4c(s3)C(=O)N(C(C(=O)Nc3nccs3)c3ccccc3)CO4)cc2)CC1 ; CN1CCC(c2ccc(-c3cc4c(s3)OCN(C(C(=O)Nc3nccs3)c3ccccc3)C4=O)cc2)CC1 ; CN1CCC(c2ccc(-c3cc4c(s3)OCN(C(C(=O)Nc3nccs3)c3ccccc3)C4=O)cc2)CC1]
or a pharmaceutically acceptable salt thereof.

53. A pharmaceutical composition comprising a compound of claim 48, or a pharmaceutically acceptable salt thereof, and at least one pharmaceutically acceptable carrier.

54. A method of inhibiting EGFR in a subject in need thereof or treating or preventing an EGFR-mediated disorder in a subject in need thereof, comprising administering to the subject a therapeutically effective amount of a compound according to claim 40.

55. A method of claim 54, wherein the EGFR-mediated disorder is a cancer selected from the group consisting of lung cancer, non-small cell lung cancer, colon cancer, breast cancer, endometrial cancer, thyroid cancer, glioma, squamous cell carcinoma, and prostate cancer.

56. A method of claim 55, further comprising the administration of an ATP-competitive EGFR inhibitor, including osimertinib, gefitinib, and elotinib, as a fixed or non-fixed combination.

57. A method of inhibiting EGFR in a subject in need thereof or treating or preventing an EGFR-mediated disorder in a subject in need thereof, comprising administering to the subject a therapeutically effective amount of a compound according to claim 48.

58. A method of claim 57, wherein the EGFR-mediated disorder is a cancer selected from the group consisting of lung cancer, non-small cell lung cancer, colon cancer, breast cancer, endometrial cancer, thyroid cancer, glioma, squamous cell carcinoma, and prostate cancer.

59. The method of claim 58, further comprising the administration of an ATP-competitive EGFR inhibitor, including osimertinib, gefitinib, and elotinib, as a fixed or non-fixed combination.
